(19) United States
(12) Patent Application Publication
(10) Pub. No.: US 2026/0265525 A1
(43) Pub. Date: Sep. 10, 2026

(54) DEVELOPMENT OF HIGHLY FLUOROGENIC STYRENE PROBES FOR VISUALIZING RNA IN LIVE CELLS

(71) Applicant: UNIVERSITY OF SOUTHERN CALIFORNIA, Los Angeles, CA (US)

(72) Inventors: Chao ZHANG, Los Angeles, CA (US); Moon Jung KIM, Los Angeles, CA (US); Yida LI, Los Angeles, CA (US)

(73) Assignee: UNIVERSITY OF SOUTHERN CALIFORNIA, Los Angeles, CA (US)

(21) Appl. No.: 19/165,975

(22) PCT Filed: Mar. 18, 2024
(86) PCT No.: PCT/US2024/020385

§ 371 Date: Sep. 16, 2025

Related U.S. Application Data
(60) Provisional application No. 63/452,996, filed on Mar. 17, 2023.

(51) Int. Cl. C09B 23/12; C12Q 1/6806

(52) U.S. Cl. CPC C09B 23/12; C12Q 1/6806

(57) ABSTRACT

A fluorogenic styrene analog probe composition for visualizing RNA includes a styrene dye. The dye includes a styrene analog having formula 1:

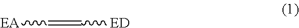

wherein the styrene analog having formula 1 is cis or trans; EA is an electron acceptor such as a pyridinium group or an isoquinolinium group; and ED is an electron donor such as an indole group or an indolizine group.

### CROSS-REFERENCE TO RELATED APPLICATIONS

This application claims the benefit of U.S. provisional application Ser. No. 63/452,996 filed Mar. 17, 2023, the disclosure of which is hereby incorporated in its(their) entirety by reference herein.

### TECHNICAL FIELD

In at least one aspect, the present invention is related to fluorogenic styrene analog probes for visualizing RNA.

### BACKGROUND

Selective labeling and visualization of cellular RNA has provided valuable knowledge and insight into RNA biology within the complex biological system.^{1, 2} Over the years, significant efforts have been directed towards developing a variety of RNA labeling methods including, but not limited to, oligonucleotide-based hybridization probes, protein-based fluorescent reporters, RNA tagging with fluorogenic aptamers, and chemo-enzymatic modification of RNA.³⁻⁸ These developments have enabled a more precise investigation of RNA localization and dynamics. However, oligonucleotide- and protein-based methods often require expensive and complicated protocols as well as additional measures to allow for the successful delivery of the probes into cells (i.e., microinjection, plasmid transfection, and/or Cu(I)-catalyzed azide-alkyne cycloaddition) thus greatly hampering their practical use for biological imaging.⁹⁻¹²

Small-molecule fluorescent dyes have emerged as powerful and versatile tools for studying biological systems.^{13, 14} Low-molecular weight dyes are easy to use and often display good cell permeability making them ideal for staining live cells without the need for fixation and permeabilization. Additionally, small molecules have superior chemical tractability and display tunable spectral and photophysical properties compared to oligonucleotide- and protein-based fluorescent probes.¹³ For these reasons, it is desirable to expand the current repertoire of fluorescent dyes for studying live cell dynamics. There are many commercially available fluorescent dyes that populate the visible spectrum and stain a variety of organelles such as the nucleus (via binding chromosomal DNA), mitochondria, and lysosomes in live cells. However, cell-permeable small-molecule dyes for imaging RNA are severely lacking.¹⁵ To date, SYTO™ RNASelect (SYTO) is the only commercially available RNA dye compatible with live-cell imaging, yet it's properties leave much to be desired.¹⁶⁻¹⁸ For instance, the blue excitation and green emission wavelengths of SYTO (λex=~490 nm; λem=~530 nm) are more scattered, more phototoxic and in regions of significant background fluorescence in biological samples than longer wavelengths. SYTO displays low photostability and limited cell permeability, which restricts its use in time-sensitive and time-dependent imaging experiments. Finally, the structure of SYTO is undisclosed and its binding mode to RNA remains elusive making it difficult to optimize its spectral and photophysical properties.

Previous efforts from two research groups have been dedicated towards the development of two indole-based “push-pull” styrene fluorophores (MPI and IN), reported to selectively stain RNA in live cells.^{19, 20} MPI (methyl pyridinium indole) and IN both contain methyl pyridinium as the electron acceptor and an indole ring as the donor (FIG. 1A). In solution, free MPI and IN are virtually non-fluorescent, producing little background fluorescence. However, upon binding RNA they exhibit greatly elevated fluorescence intensity. This attribute of “turn-on” fluorescence is particularly advantageous for sensing and imaging because fluorescence is only activated upon interaction with their biological targets, resulting in low background signals and high-contrast images.²¹ MPI and IN absorb and emit in the blue-green range of the visible spectrum (~440 nm and ~540 nm) with the former reported to have a fluorescence quantum yield of 32%, roughly double that of SYTO (17%). Although MPI was found to display more favorable photophysical properties (e.g., higher photostability and larger stokes shift) compared to SYTO, it was still difficult to generate high-contrast images because of its low quantum yield.

Accordingly, attracted by the small size and RNA selectivity of MPI-like dyes, there is a need to improve the quantum yield, photostability, fluorogenic response, and emission wavelength of styrene dyes through chemical modifications.

### SUMMARY

In at least one aspect, a fluorogenic styrene analog probe dye composition for visualizing RNA is provided. The fluorogenic styrene analog probe composition includes a styrene analog having formula 1:

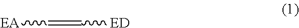

wherein:
the styrene analog having formula 1 is cis or trans;
EA is an electron acceptor selected from the group consisting of:

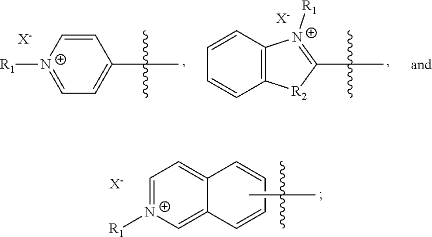

ED is an electron donor selected from the group consisting of:

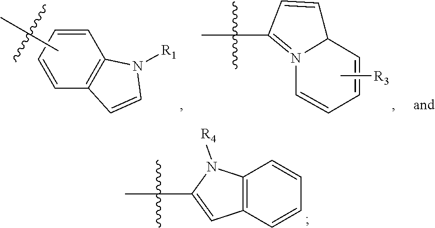

R₁ is H or C₁₋₈ alkyl;
R₂ is S or C(R₅)₂;
R₃ is H, C₁₋₈ alkyl, halo, C₁₋₈ alkoxy, —NH₂, —N(R₃)₂, or piperidinyl; and
R₄ is H or C₁₋₈ alkyl; and
R₅ is C₁₋₈ alkyl

In another aspect, the fluorogenic styrene analog probe dye composition provides highly sensitive and rapid staining.

In another aspect, the fluorogenic styrene analog probe dye composition provides bright imaging (with improved quantum yields).

In another aspect, the fluorogenic styrene analog probe dye composition provides red-shifted absorption and emission.

In another aspect, the fluorogenic styrene analog probe dye composition provides an excellent fluorogenic response (large fold increases upon binding to RNA)

In another aspect, the fluorogenic styrene analog probe dye composition is compatible with multiple imaging modes (i.e., confocal fluorescence, FLIM)

In another aspect, the fluorogenic styrene analog probe dye composition provides high solubility (>100 uM in PBS, unlike typical hydrophobic fluorescent dyes)

In another aspect, the fluorogenic styrene analog probe dye composition is low cost.

Styrene dyes are useful imaging probes and fluorescent sensors due to their strong fluorogenic responses to environmental changes or binding to macromolecules. Previously, indole-containing styrene dyes have been reported to selectively bind RNA in the nucleolus and cytoplasm. However, the application of these indole-based dyes in cell imaging is limited by their moderate fluorescence enhancement and quantum yields, as well as relatively high background associated with these green-emitting dyes. In this work, the positional and electronic effects of the electron donor by generating regioisomeric and isosteric analogs of the indole ring are investigated. Select probes exhibited large stokes shifts, enhanced molar extinction coefficients, and bathochromic shifts in their absorption and fluorescence wavelengths. In particular, the indolizine analogs displayed high membrane permeability, strong fluorogenic responses upon binding RNA, compatibility with fluorescence lifetime imaging microscopy (FLIM), low cytotoxicity, and excellent photostability. These indolizine dyes not only give rise to rapid, sensitive, and intense staining of nucleoli in live cells but can also resolve sub-nucleolar structures, enabling highly detailed studies of nucleolar morphology. Furthermore, the novel dyes can partition into RNA coacervates and resolve the formation of multiphase complex coacervate droplets. These indolizine-containing styrene probes offer the highest fluorescence enhancement among the RNA-selective dyes reported in the literature; thus, these new dyes are excellent alternatives to the commercially available RNA dyes, such as SYTO™ RNASelect, for visualizing RNA in live cells and in vitro.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1A: Chemical structures of the previously reported styrene-based RNA-selective dyes, MPI and IN.

FIG. 1B: Chemical structures of the new dyes 1a-1m reported herein.

FIG. 1C: Scheme 1. Synthesis of the styrene dyes 1a-1d.

FIG. 1D-1E: Table 1. ^{a}DMSO: dimethyl sulfoxide. T.E. buffer: 10 mM Tris-HCl, 1 mM EDTA, pH=7.5. RNA: 200 μg/mL type IV torula yeast. ^{b}Absorbance maximum wavelength (nm). ^{c}Molar absorptivity (M-1 cm-1). ^{d}Fluorescence maximum wavelength (nm). ^{e}Fluorescence quantum yield of dyes using Coumarin 6 (Φ500=0.78) and Rhodamine 6G (Φ548=0.95). Fluorescence quantum yield of dyes incubating with nucleotides using integrating sphere. Quantum yield data were averaged from duplicate experiments.

FIGS. 2A, 2B, 2C, 2D, 2E, and 2F: Absorption and emission spectra of (A) 1c (B) 1d and (C) MPI in T.E. buffer (pH 7.5) or RNA (Type IV RNA from torula yeast; 200 g/mL). Dye concentration: 10 μM. The dashed and solid lines indicate absorbance and emission, respectively. (D) Comparison of fluorescence intensity between MPI, 1c, and 1d in buffer vs. dyes in RNA solution (200 g/mL) dye concentration 10 M. The dashed lines reflect the fluorescence intensity of unbound dye, and the solid lines indicate the fluorescence intensity of dyes in RNA solution. (E) Fluorescence titration of 1c (10 μM) incubated with increasing concentration of nucleic acids or protein (BSA; bovine serum albumin). (F) Measurements of fluorescence enhancement.

FIG. 3: Confocal fluorescence images of live HeLa cells incubated with 20 M MPI (λₑₓ=470 nm, λₑₘ=525-555 nm), 1c (λₑₓ=550 nm, λₑₘ=580-620 nm), and 1d (λₑₓ=500 nm, λₑₘ=560-600 nm) for 30 min. TPC: transmission phase contrast. Scale bar: 10 μm.

FIGS. 4A, 4B, and 4C: (A) Confocal fluorescence images of PFA-fixed HeLa cells incubated with 20 μM of 1c and 1d for 30 min and 1 μg/mL Hoechst 33342 for 30 min. Scale bar: 10 μm. (B) Zoomed in image of HeLa cells stained with 20 μM of 1c for 30 min. Image shows the sub-nucleolar components FC (indicated by the white arrowhead), DFC, and GC (indicated by the white arrows). TPC: transmission phase contrast. Scale bar: 3 μm. (C) Confocal fluorescence images of live HeLa incubated with 10 μM of MPI, 1a, 1e, 1h, 1f, and 1g for 30 min.

FIG. 5: Comparison of intensity and FLIM phasor processed images of HeLa cells stained with 1c (20 μM). Separation of lifetimes was performed using FLIM phasor analysis, in which very short lifetimes appear on the lower right and very long lifetimes appear on the lower left. The phasor plot shows four unique lifetimes (color-coded on the FLIM image), corresponding to fluorophores within the nucleolus, nucleus, cytoplasm, and surrounding media. Scale bar: 10 μm.

FIG. 6: Comparison of photobleaching of SYTO™ RNASelect and 1c. Confocal fluorescence images of PFA-fixed HeLa cells incubated with 1c (1 μM) and SYTO™ RNASelect (1 μM). Scale bar: 20 μm.

FIG. 7: Partitioning of 1c (10 μM) within the RNA/spermine condensates. The top row shows images enlarged by 1× while the bottom row shows images enlarged by a factor of 8×. The left column shows images based on fluorescence intensity alone and the middle column shows images resolved using FLIM. The phasor plot indicates the presence of a single lifetime species of 1c (2.6 ns). Scale bar: 10 μm.

FIGS. 8A, 8B, 8C, 8D, 8E, 8F, 8G, 8H, 8I, 8J, 8K, 8L, and 8M. Reason schemes for the dyes set forth herein.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: all R groups (e.g. Rᵢ where i is an integer) include hydrogen, alkyl, lower alkyl, C₁₋₆ alkyl, C₆₋₁₀ aryl, C₆₋₁₀ heteroaryl, —NO₂, —NH₂, —N(R′R″), —N(R′R″R′″)L⁻, Cl, F, Br, —CF₃, —CCl₃, —CN, —SO₃H, —PO₃H₂, —COOH, —CO₂R′, —COR′, —CHO, —OH, —OR′, —O-M⁺, —SO₃⁻M⁺, —PO₃-M⁺, —COO⁻M⁺, —CF₂H, —CF₂R′, —CFH₂, and —CFR′R″ where R′, R″ and R′″ are C₁₋₁₀ alkyl or C₆₋₁₉ aryl groups, L⁻ is a negative counter ion, and M⁺ is a positive counterion; single letters (e.g., “n” or “o”) are 1, 2, 3, 4, or 5; in the compounds disclosed herein including compounds described by formula or by name, a CH bond can be substituted with alkyl, lower alkyl, C₁₋₆ alkyl, C₆₋₁₀ aryl, C₆₋₁₀ heteroaryl, —NO₂, —NH₂, —N(R′R″), —N(R′R″R′″)L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁻, —COO⁻M⁺, —CF₂H, —CF₂R′, —CFH₂, and —CFR′R″ where R′, R″ and R′″ are C₁₋₁₀ alkyl or C₆₋₁₈ aryl groups, L⁻ is a negative counter ion, and M⁻ is a positive counterion; percent, “parts of,” and ratio values are by weight; the term “polymer” includes “oligomer,” “copolymer,” “terpolymer,” and the like; molecular weights provided for any polymers refer to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

The term “alkyl” refers to C₁₋₂₀ inclusive, linear (i.e., “straight-chain”), branched, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g. pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

Throughout this application, where publications are referenced, the disclosures of these publications in their entireties are hereby incorporated by reference into this application to more fully describe the state of the art to which this invention pertains.

### Abbreviations

“FLIM” means fluorescence lifetime imaging microscopy.

“IN” means indole.

“MPI” means methyl pyridinium indole.

“PBS” means phosphate buffered saline.

In a first embodiment, a fluorogenic styrene analog probe composition for visualizing RNA comprising a styrene analog having formula 1:

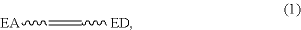

where EA is an electron acceptor and ED is an electron donor. It should be appreciated that formula 1 includes both cis and trans isomers of the double bond as illustrated by formula 1a and 1b:

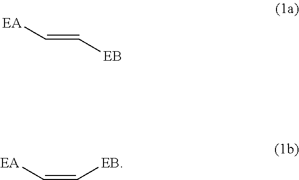

Moreover, it should also be appreciated that the styrene analogs are dyes.

In another aspect of the first embodiment, EA is an electron acceptor selected from the group consisting of:

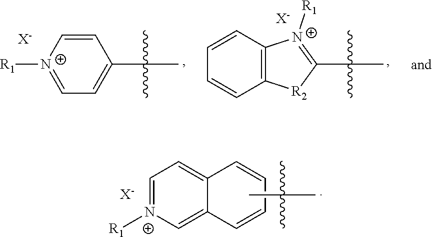

In another aspect of the first embodiment, ED is an electron donor selected from the group consisting of:

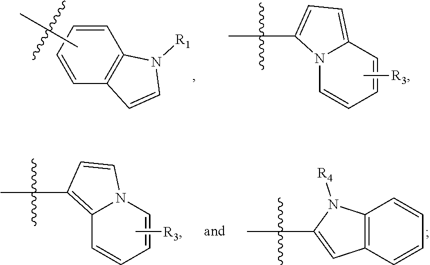

wherein:
R₁ is H or C₁₋₈ alkyl;
R₂ is S or C(R₅)₂;
R₃ is H, C₁₋₈ alkyl, halo, C₁₋₈ alkoxy, —NH₂, —N(R₃)₂, or piperidinyl; and
R₄ is H or C₁₋₈ alkyl; and
R₅ is C₁₋₈ alkyl.

In another aspect of the first embodiment, EA is an electron acceptor having the following formula:

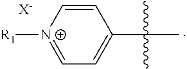

In another aspect of the first embodiment, the fluorogenic styrene analog probe composition includes comprising a component selected from the group consisting of:

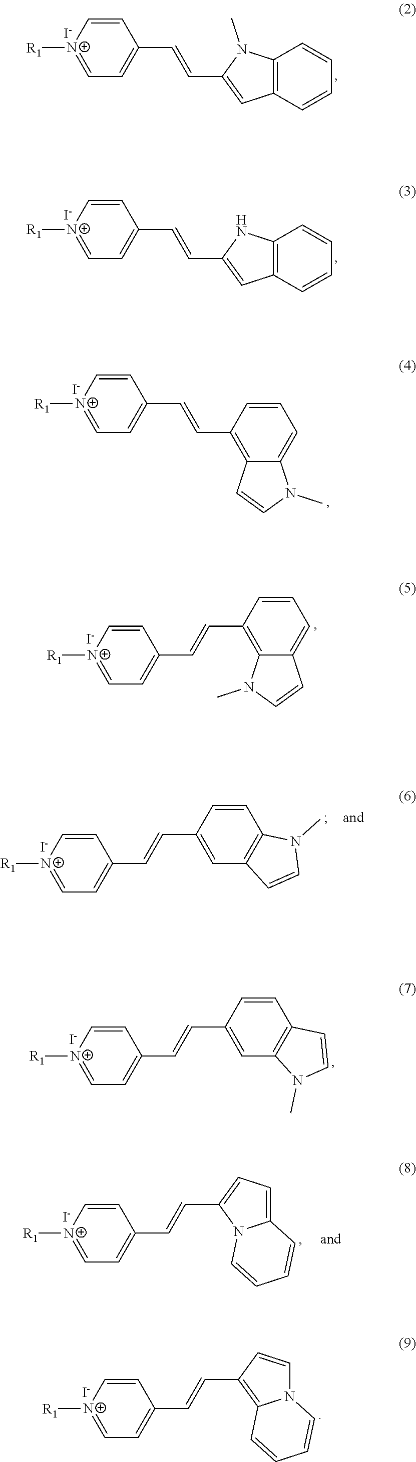

In another aspect of the first embodiment, EA is an electron acceptor having the following formula:

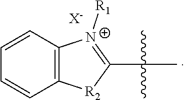

In another aspect of the first embodiment, the fluorogenic styrene analog probe composition includes a component selected from the group consisting of:

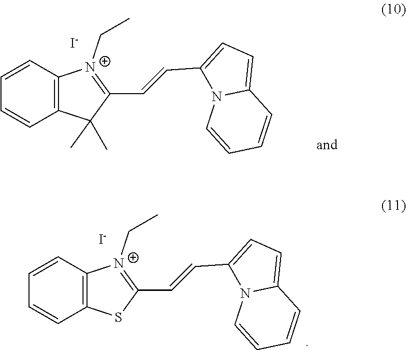 [SMILES: CC[N+]1=C(/C=C/c2ccc3ccccn23)C(C)(C)c2ccccc21 ; [I-] ; CC[n+]1c(/C=C/c2ccc3ccccn23)sc2ccccc21 ; [I-]]

In a second embodiment, a fluorogenic styrene analog probe composition for visualizing RNA comprising a styrene analog having formula 1:

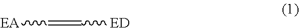

wherein:
the styrene analog having formula 1 is cis or trans;
EA is an electron acceptor selected from the group consisting of:

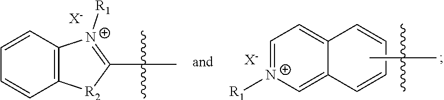

ED is an electron donor having the following formula:

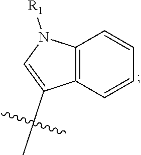

R₁ is H or C₁₋₈ alkyl, R₂ is S or C(R₅)₂;
R₃ is H, C₁₋₈ alkyl, halo, C₁₋₈ alkoxy, —NH₂, —N(R₃)₂, or piperidinyl; and
R₅ is C₁₋₈ alkyl.

In another aspect of the second embodiment, EA is an electron acceptor having the following formula:

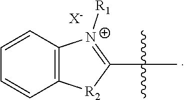

In another aspect of the second embodiment, the fluorogenic styrene analog probe composition a compound having the following formula:

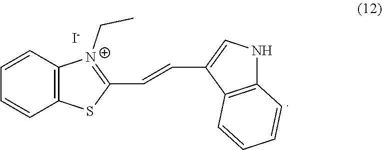 [SMILES: CC[n+]1c(/C=C/c2c[nH]c3ccccc23)sc2ccccc21 ; [I-] ; "."]

In another aspect of the second embodiment, the fluorogenic styrene analog probe composition includes EA is an electron acceptor having the following formula:

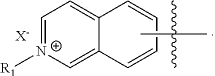

In another aspect, of the second embodiment, the fluorogenic styrene analog probe composition includes a component selected from the group consisting of:

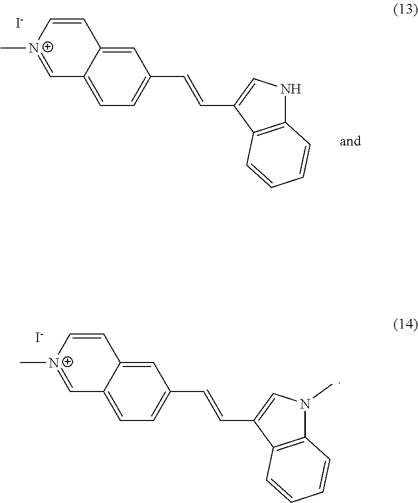

In another aspect, the fluorogenic styrene analog probe composition further includes a solvent. In a refinement, the solvent is a polar solvent such as water (e.g., a buffer solution), alcohols (e.g., methanol, ethanol, propanol), dimethyl sulfoxide, dimethylformamide, acetonitrile, N,N-Dimethylacetamide, tetrahydrofuran, dichloromethane, ethylene glycol, propylene carbonate, and the like. Typically, the styrene analog has a solubility greater than 1 μM in the solvent. In a refinement, the styrene analog has a solubility from 1 μM to 50 μM in the solvent. In some refinements, the styrene analog has a solubility greater than, in increasing order of preference, 10 nM, 1 μM, 5 μM, 10 μM, 30 μM, 50 μM, or 100 μM in the solvent. Although the not limited by maximum solubility, the styrene analog typically has a solubility less than about 1000 μM in the solvent.

In another aspect, the fluorogenic styrene analog probe composition provides red-shifted absorption and emission styrene-based relative to prior art fluorescent molecules, and specifically, relative to MPI (methyl pyridinium indole) and related compounds. These shifts represent an enhancement in the photophysical properties of the dyes, making them more suitable for visualizing RNA within live cells with improved quantum yields, photostability, and a substantial increase in fluorescence enhancement upon binding RNA.

In another aspect, the styrene analogs display high membrane permeability and strong fluorogenic responses upon binding RNA. This property is crucial for live-cell imaging applications where the probe must efficiently penetrate cell membranes and exhibit a significant increase in fluorescence upon binding its target RNA.

In another aspect, the probe compositions (and therefore, the styrene analogs are compatible with FLIM, allowing for selective imaging based on fluorescence lifetimes. This enables the resolution of cellular structures beyond fluorescence intensity and emission wavelength, offering a more detailed view of cellular dynamics and the spatial distribution of RNA.

In another aspect, the probe compositions demonstrate low cytotoxicity, making them suitable for long-term imaging studies without adversely affecting cell viability. Additionally, their excellent photostability minimizes photobleaching, enabling extended observation periods and reducing the likelihood of data loss over time.

In another aspect, the probe compositions allow for rapid and sensitive staining of nucleoli and can resolve sub-nucleolar structures, facilitating highly detailed studies of nucleolar morphology. This feature is particularly valuable for investigating the roles of nucleoli in cellular processes.

In another aspect, the probe compositions can partition into RNA coacervates and resolve the formation of multiphase complex coacervate droplets. This ability is significant for studying liquid-liquid phase separation (LLPS) and the formation of membrane-less organelles, providing insights into the molecular basis of various biological processes and diseases.

In another aspect, the styrene analogs (i.e., the dyes exhibit high sensitivity and specificity for RNA, with concentrations as low as 1 nM successfully used to resolve nucleoli. This high specificity is critical for accurately visualizing RNA without interference from other cellular components.

In another aspect, the probe compositions are compatible with other red-colored dyes (λₑₘ>580 nm) regardless of spectral overlap. This compatibility allows for multiplexing with other dyes to simultaneously visualize different cellular components or processes.

In another embodiment, a method for imaging RNA is a biological sample using the compositions set forth herein is provided. The method includes the step of contacting the biological sample with a fluorogenic styrene analog probe composition for visualizing RNA to form a stained sample. In a refinement, the biological sample is selected from a group consisting of cultured cell lines, tissue sections, and single-cell suspensions. Typically, the fluorogenic styrene analog probe composition is applied to the biological sample at a concentration ranging from 1 μM to 50 μM.

The fluorogenic styrene analog probe composition is as described above. A fluorescence imaging technique is applied to visualize the stained sample. Characteristically, the fluorescence imaging technique includes using a specific wavelength for excitation and emission that maximizes the fluorescence signal from the fluorogenic styrene analog probe. In one variation, the fluorescence imaging technique is selected from the group consisting of confocal microscopy, two-photon microscopy, fluorescence lifetime imaging microscopy (FLIM), and super-resolution microscopy. In a refinement, the fluorescence imaging technique is confocal fluorescence microscopy. In another refinement, the fluorescence imaging technique is fluorescence lifetime imaging microscopy. Advantageously, the method is compatible with live- and fixed-cell imaging. In a refinement, the method further includes a step of screening for compounds that affect RNA metabolism, stability, or localization in live cells. In a refinement, the fluorogenic styrene analog probe is incubated with the biological sample for a period ranging from 5 to 60 minutes before imaging. In a variation, the method further include a washing step after the contacting step to remove unbound fluorogenic styrene analog probe from the biological sample before applying the fluorescence imaging technique. In a variation, the method further includes a step of quantifying the RNA based on the fluorescence intensity measured from the stained sample.

In another aspect, a method for visualizing RNA in live cells is provided. The method includes steps of providing a live cell sample and staining the live cell sample with a fluorogenic styrene analog probe composition as set forth above In a refinement, the probe concentration is greater than 1 μM as set forth above, the live cell samples are incubated with the fluorogenic styrene probe for a first predetermined time (e.g., about 30 minutes). The method further includes a step of imaging the live cell sample using fluorescence microscopy to visualize RNA within the live cells. In a refinement, the fluorescence microscopy is selected from a group consisting of confocal microscopy, two-photon microscopy, and fluorescence lifetime imaging microscopy (FLIM). In a refinement, the method further incudes a step of washing the live cell sample after incubation and before imaging to remove unbound fluorogenic styrene probes.

In another embodiment, a live-cell RNA imaging kit is provided. The live-cell RNA imaging kit includes one or more fluorogenic styrene probes as defined above. In a refinement, the live-cell RNA imaging kit includes instructions for staining live cells with the fluorogenic styrene probes and guidelines for fluorescence imaging, including suggested incubation times (e.g., at least 5 minutes) and concentrations as set forth above for optimal RNA visualization. In a refinement, the live-cell RNA imaging kit includes a set of control probes that do not bind RNA (e.g., phosphate-buffered saline) for use as negative controls in fluorescence imaging.

In another embodiment, a method for assessing RNA localization and dynamics in live cells is provided. The method includes a step of staining live cells with a fluorogenic styrene probe according to the method for visualizing RNA in live cells and the fluorogenic styrene analog probe compositions as set forth above. Fluorescence lifetime imaging microscopy (FLIM) is utilized to measure fluorescence lifetimes indicative of RNA localization and dynamics within the cells. In a refinement, fluorescence lifetime imaging microscopy (FLIM) utilizes time-correlated single-photon counting (TCSPC) to measure the fluorescence lifetimes.

Additional details of the invention are found in Moon Jung Kim, Yida Li, Jason A. Junge, Nathan K. Kim, Scott E. Fraser, and Chao Zhang. Development of Highly Fluorogenic Styrene Probes for Visualizing RNA in Live Cells. ACS Chemical Biology 2023 18 (7), 1523-1533. DOI: 10.1021/acschembio.3c00141 and its supplemental information; the entire disclosure of which is hereby incorporated by reference.

The following examples illustrate the various embodiments of the present invention. Those skilled in the art will recognize many variations that are within the spirit of the present invention and scope of the claims.

### 1. Introduction

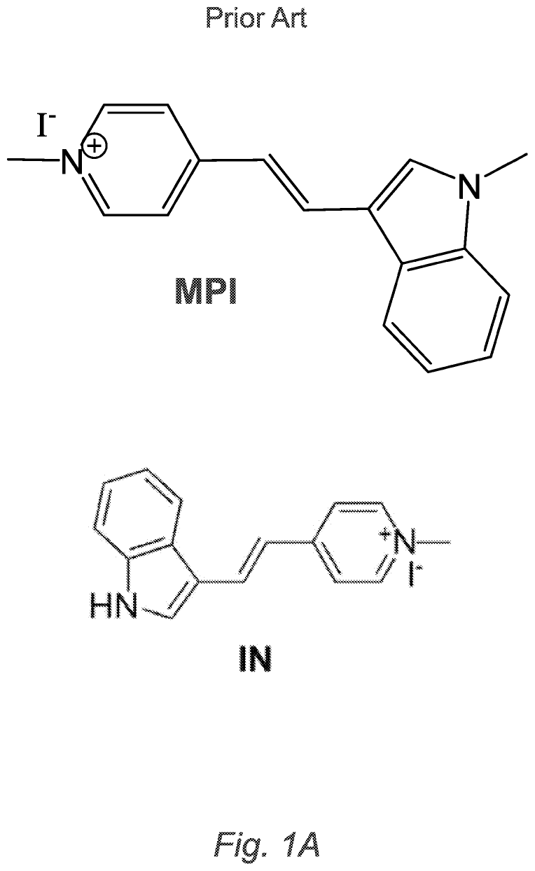

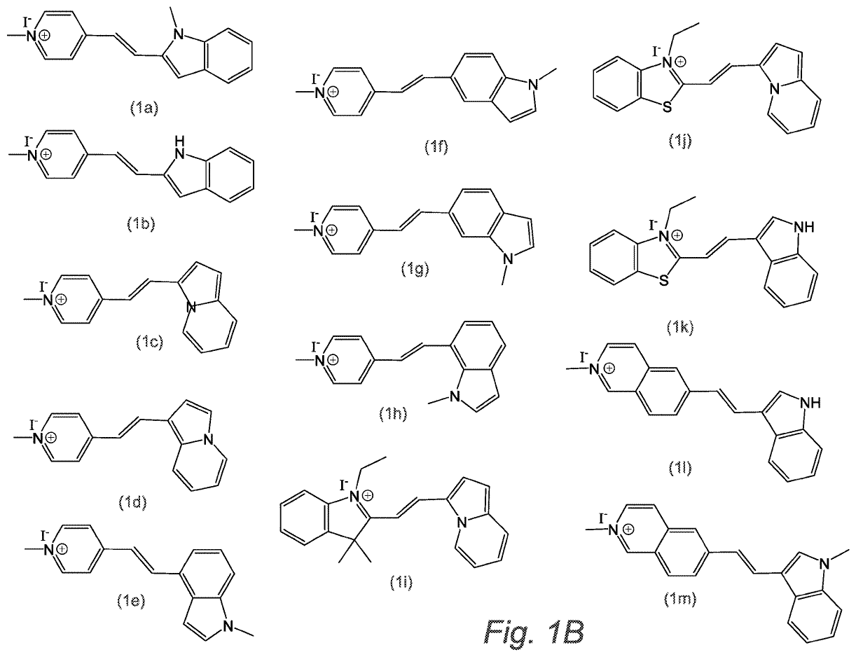

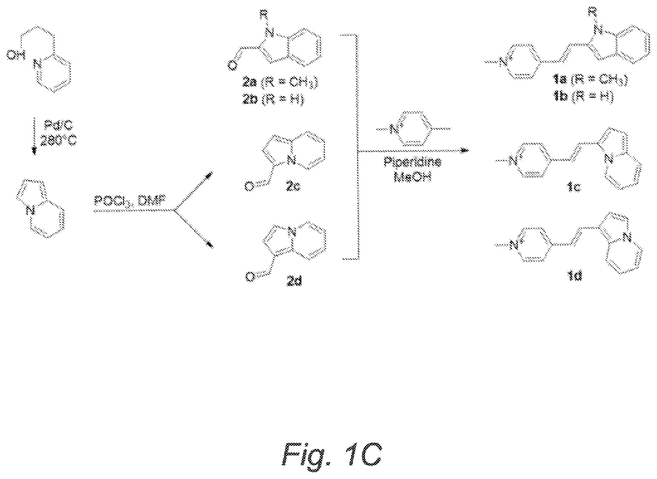

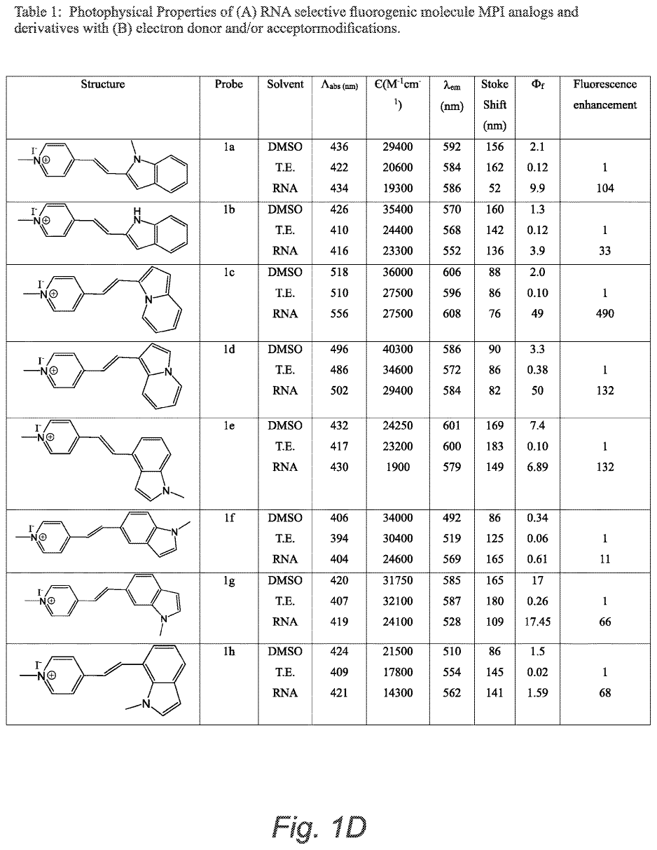

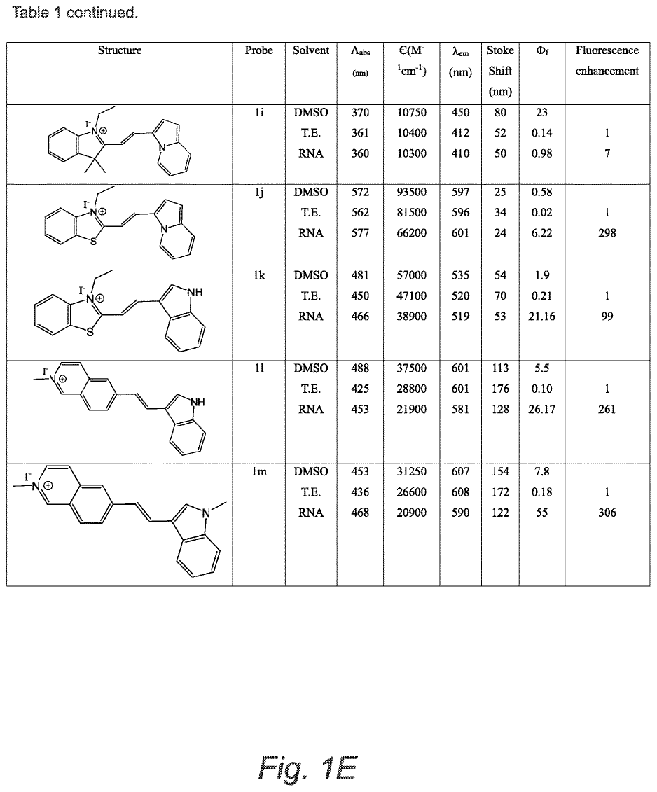

In general, the spectral and photophysical properties of styrene dyes, such as MPI and IN, can be improved via modification of the indole donor. Herein, this hypothesis is evaluated by synthesizing and characterizing a panel of novel styrene dyes, which consist of regioisomeric and isosteric analogs of MPI and IN (FIG. 1C/1A). The new styrene analogs not only retain their turn-on fluorescence upon binding RNA, but also demonstrate red-shifted absorption and emission wavelengths. In particular, two indolizine-containing dyes displayed improved quantum yields, high photostability, and substantially greater fluorescence enhancement (>7-fold and >2-fold increase) upon binding RNA compared to MPI. These indolizine-containing dyes are also compatible with fluorescence lifetime imaging (FLIM) and can be used to resolve nucleoli in cells and multiphase separation in RNA coacervates.

### 2. Results and Discussion

2.1 Design and Synthesis of Dyes 1a-1m

MPI and IN were used as the parental compounds in the design of new styrene dyes (FIG. 1A). Two strategies to modify the indole donor are utilized. One strategy involved retaining the indole donor and changing the substitution position on the indole ring while the other involved replacing the indole donor or the pyridinium acceptor with alternative donors or acceptors such as the indolizine, an isostere of the indole. These strategies led to the design of analogs (1a through 1m) provided in FIG. 1B.

The synthesis of compounds 1a-1d is illustrated in Scheme 1 (FIG. 1C). Indolizine was synthesized through a palladium-catalyzed intramolecular cyclization of 2-pyridinepropanol, and subsequently converted into the formyl indolizine intermediates (2c and 2d) using the Vilsmeier-Haack reaction based on previously reported procedures.22, 23 In the final step, the formyl-indoles (2a and 2b) and formyl-indolizines (2c and 2d) underwent a Knoevenagel condensation reaction with N-methyl-4-methylpyridinium to yield the styrene probes 1a-1d. The products were characterized by ¹H-NMR, ¹³C-NMR and MS with all the spectroscopic data in agreement with the proposed chemical structures.

2.2 Spectral and Photophysical Characterization of 1a-1m

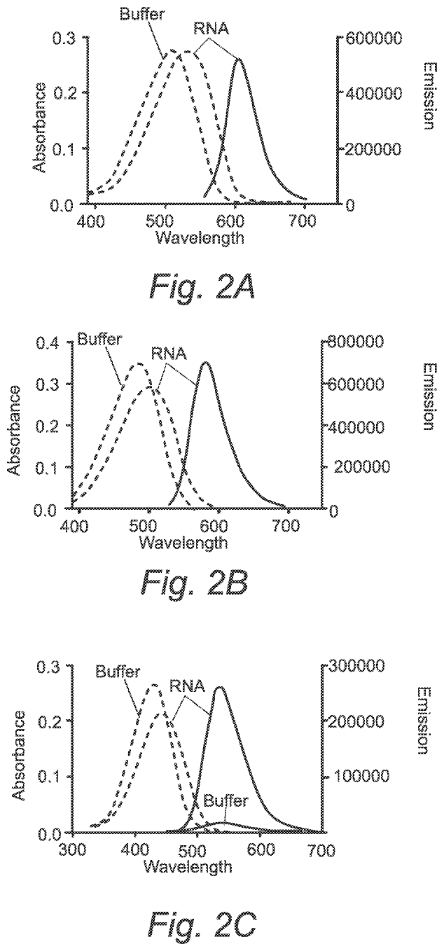

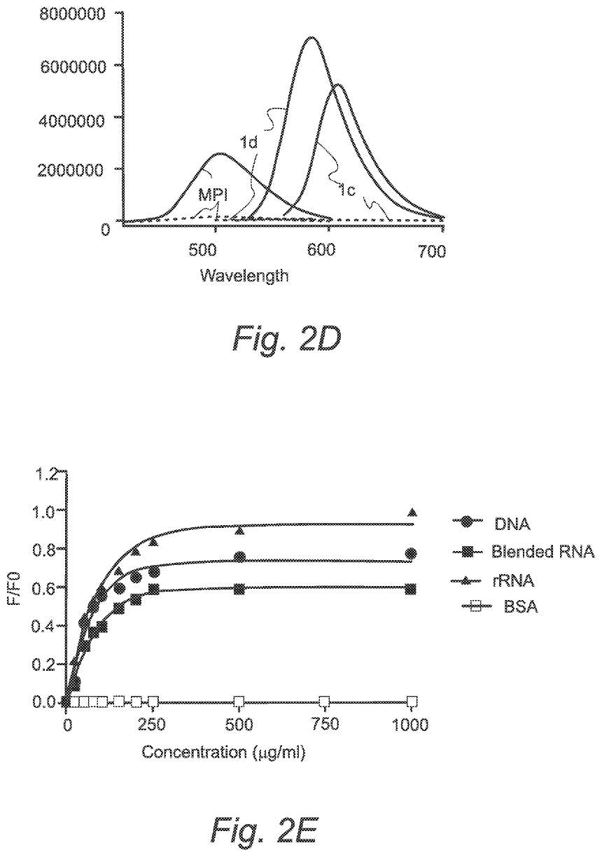

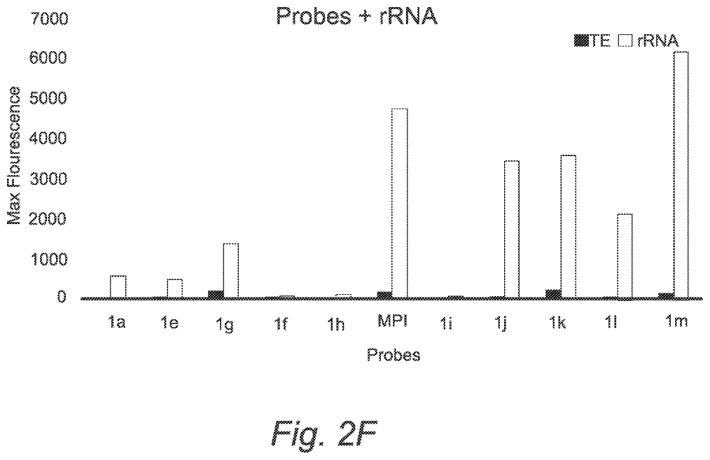

After the synthesis of the styrene dyes, their photophysical properties were evaluated. The spectral and photophysical properties (absorption, emission, molar absorptivity (ε), fluorescence quantum yield (ΦDF), and fluorescence enhancement) of probes 1a-1d were measured in DMSO and T.E. buffer (Tris-HCl, 1 mM EDTA, pH=7.5). The absorbance and emission spectra of 1a-1d are shown in FIGS. 2A-C, and their photophysical properties are summarized in Table 1 (FIGS. 1D and 1E). FIG. 2D provides measurements of the fluorescent enhancement for several of the fluorescent probes.

Slight solvatochromic shifts in the absorbance and emission spectra of 1a-1d in DMSO and T.E buffer are observed. These dyes are slightly more red-shifted in DMSO and RNA than in T.E. buffer. In T.E. buffer, 1a and 1b exhibited maximum absorbance wavelengths at 422 and 410 nm and a broad emission band centered at 584 nm and 568 nm, respectively, indicative of a charge transfer excited state.²⁴ There are slight differences in the absorption and emission wavelengths of 1a and 1b, consistent with the minor structural and electronic differences between the two analogs. Dyes 1a and 1b exhibited large Stokes shifts of 162 and 142 nm with molar absorptivity values of 20600 and 24400 M⁻¹ cm⁻¹. As expected, the fluorescence quantum yields of the free dyes in solution were low (0.12% for 1a and 1b) which is consistent with the ability of styrene dyes to dissipate energy through rotation leading to nonradiative relaxation to the ground state.²⁵⁻²⁷ Direct comparison of the photophysical properties of regio-isomers MPI and 1a showed that both dyes shared similar absorption wavelengths while the emission wavelength of 1a was significantly red-shifted (>50 nm) compared to that of MPI. The differences in the photophysical properties observed for the indole regio-isomers may be explained by how electrons move through the conjugated n-system between the two resonance forms of the styrene dyes. Such qualitative analysis reveals that the electron transfer within 1a and 1b spans larger conjugated π-systems than that of MPL Thus, the conjugated π-system of the 2-indole derivatives participates in electron transfer to a higher degree than those of the 3-indole derivates. This may account for the observed, red-shifted emission of 1a and 1b compared to MPI.

The indolizine dyes 1c and 1d displayed a significant red shift in both absorption (510 and 486 nm) and emission (596 and 572 nm) wavelengths (FIG. 1D). The size difference between the conjugated π-system undergoing electron movement may account for the observed bathochromic shift of the indolizine dyes. Dyes 1c and 1d show narrow emission bands and moderate solvatochromic effects which supports that emission occurs from a locally excited state.28, 29 1c and 1d displayed a large Stokes shift of 86 nm and molar absorptivity values of 27500 and 34600 M-1 cm-1. As expected, the fluorescence quantum yields of the free dyes in T.E. buffer were low, with values at 0.10% and 0.38% for 1c and 1d, respectively.

To determine the fluorescence change of 1a-1d in the presence of RNA, the dyes (10 μM) were incubated with torula yeast type IV RNA in T.E. buffer and measured the spectral and photophysical parameters as described above. Fold-enhancement values were calculated by taking the ratio of the quantum yield of dyes in RNA solution over free dyes. Dyes 1a and 1b exhibited an 104- and 33-fold fluorescence enhancement with modest quantum yields of 9.9% and 3.9%, respectively (FIG. 1D). While 1a displayed a similar degree of fluorescence enhancement compared to MPI, 1b displayed only a 33-fold fluorescence enhancement, roughly a third of the observed fluorescence enhancement of MPI. This data suggests that a methyl substituent on the indole nitrogen can enhance quantum yields possibly due to the electron-donating effect imparted by the methyl group.

The indolizine dyes 1c and 1d showed a significant increase in fluorescence intensity upon addition of RNA with absolute quantum yield values of 49% and 50% (FIG. 1D). A remarkable 490- and 132-fold fluorescence enhancement of 1c and 1d in response to binding RNA is observed. Notably, the quantum yield and fluorogenic response of indolizine-containing dyes (1c and 1d) are much higher than those of indole-containing dyes (1a, 1b, and MPI). Given that 1c and 1d display the most favorable spectral and photophysical qualities among the styrene dyes, it was decided to further characterize these indolizine dyes in subsequent in vitro and cell imaging experiments. When 1c was incubated with DNA, it generated a lower fluorogenic response than RNA (FIG. 2E), indicating a moderate selectivity for RNA over DNA in vitro. Upon incubation with the same RNA species, 1c demonstrated significantly higher fluorogenic responses than SYTO™ RNASelect. Additionally, it was confirmed that 1c can stain RNA in PAGE-gels, although its sensitivity was lower than the commercial standard, SYBR Gold. It is speculated that the smaller size of 1c than commercial cyanine dyes, such as SYBR Gold, may be responsible for its comparatively lower sensitivity in vitro when staining RNA in gels.

2.3 Live- and fixed-cell imaging using 1c and 1d

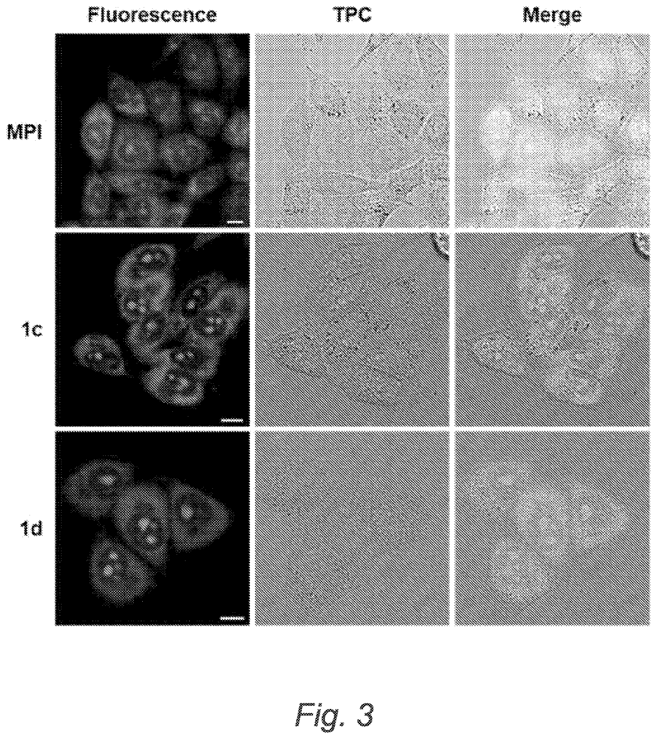

To determine the cellular localization of 1c and 1d, live HeLa cells were stained with 20 μM of either dye for 30 min prior to image acquisition. Fluorescence signals of 1c and 1d can be seen primarily within the nucleoli and cytoplasm (FIG. 3), and the nucleus is otherwise dark, consistent with preferential binding the dyes to RNA. The localization of 1c and 1d were similar to HeLa cells stained with MPJ. The intensity and contrast of HeLa cell images obtained using 1c or 1d are substantially higher than those of MPI, consistent with the higher quantum yield and fold-enhancement observed with the indolizine dyes (Table 1). Viewing the fluorescence images of 1c and 1d at higher magnification, distinguishable fiber-like structures distributed throughout the cytoplasm that resemble mitochondrial staining profiles are observed. This can be explained by the lipophilic, cationic character of 1c and 1d, which allow for accumulation in the mitochondria similar to established mitochondrial dyes (e.g., MitoView™ and MitoTracker™) due to the negative mitochondrial membrane potential.^{30,31}

The new dyes displayed excellent kinetics and sensitivity for imaging nucleoli in live cells. Nucleoli could be clearly discerned as early as 30 seconds after addition of either 1c or 1d in the medium, while no signals from SYTO could be detected even after 30 minutes of incubation following the manufacturer's protocol. A dose-response study revealed that nanomolar concentrations of 1c were sufficient for visualizing nucleoli in live HeLa cells.

To determine whether dyes 1c and 1d show similar localization profiles under fixed conditions, HeLa cells were fixed with 4% paraformaldehyde (PFA) before staining with either dye. It was found that 1c and 1d show qualitatively similar labeling patterns as observed with live-cell imaging, with fluorescence signals predominantly coming from the nucleoli and cytoplasm. As expected, fixation abolished the mitochondrial membrane potential and the fiber-like appearance of the staining was absent in the cytoplasm. The nucleolar staining of 1c was confirmed to be RNA-dependent via an experiment in which fixed cells were treated with RNase.

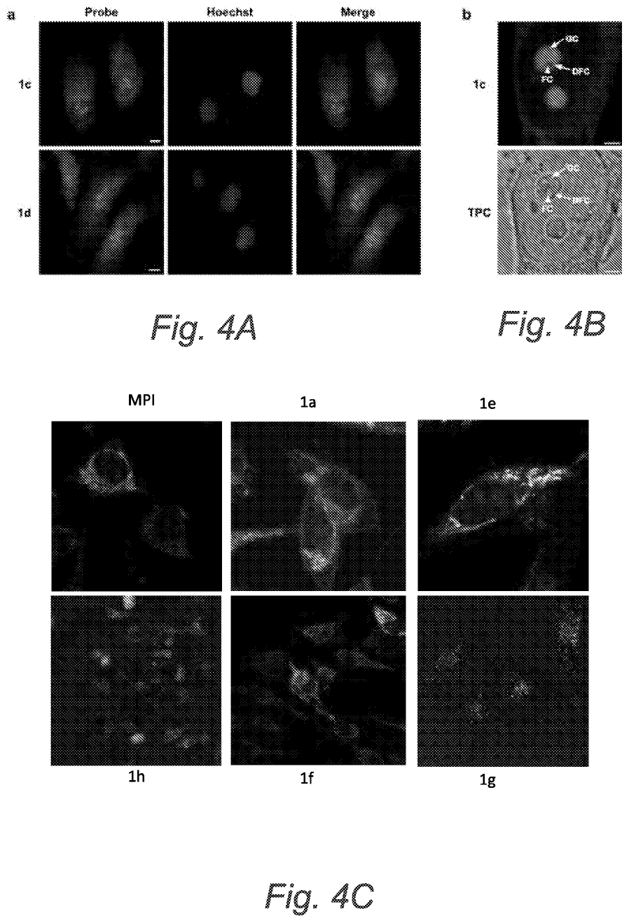

To assess the counterstaining compatibility of probes 1c and 1d, HeLa cells were stained with either 1c or 1d and Hoechst 33342 (FIG. 4A). Fluorescence signals from the dyes can be clearly seen within nucleolar and cytoplasmic structures, while Hoechst 33342 signals are restricted within the nucleus, as expected. The data suggest that fluorescence signals from 1c-1d and Hoechst 33342 are distinct and that the two types of dyes are compatible in co-staining experiments.

Next, it was investigated whether the indolizine dyes could resolve the substructures of the nucleolus. Morphologically, the nucleolus is divided into a fibrillar center (FC), a dense fibrillar component (DFC) and an outer granular component (GC) all with distinct functions in ribosome biogenesis.³² Enlarged images of the nucleolus clearly show 1c is able to reveal distinct sub-nucleolar regions by fluorescence microscopy (FIG. 4B). The images show the GC intensely stained by 1c while the FC and DFC appear as much dimmer cavities within the nucleolus. These observations can be attributed to the presence of RNA within the sub-nucleolar structures. The GC, rich in rRNA and ribosomal proteins, contain pre-ribosomal subunits while the FC contains clusters of condensed rDNA chromatin. ³³, The fluorescence images obtained using 1c are corroborated by transmission phase contrast, in which the nucleolar structures can be discerned albeit with lower resolution (FIG. 4B).^{32, 35} These data suggest that 1c can be used to visualize and study sub-nucleolar structures and nucleolar morphology in live and fixed cells. FIG. 4C provides confocal fluorescence imaging of live Hela cells with 30 mins cultured with styrene dyes comparing MPI, 1a, and 1e

To further characterize the novel dyes in cells, Fluorescence Lifetime Imaging Microscopy (FLIM) was performed to study whether the dyes display unique fluorescence lifetimes in cells. Four distinct fluorescence lifetime species of 1c were observed depending on its sub-cellular locations: nucleolus (3.4 ns), nucleus (2.5 ns), cytoplasm (1.6 ns), and unbound dye (0.82 ns). The phasor approach was utilized to microenvironment through a graphical interface with imaging data.^{36, 37}. FIG. 5 shows a side-by-side comparison of intensity-based and FLIM phasor masked-images, highlighting differences in subcellular RNA-positive microenvironments. Dyes like 1c with unique and separable lifetimes are valuable tools for differentiating multiple RNA-containing structures that are stained by the same dyes and for monitoring the dynamics of the RNA-containing structures at those locations. These data suggest that FLIM can provide a second dimension to distinguish the different cellular structures stained by the same indolizine dye. FLIM analysis, as it provides a powerful fit-free tool to characterize and display differences in dye.

### 2.4 Cytotoxicity and Photostability

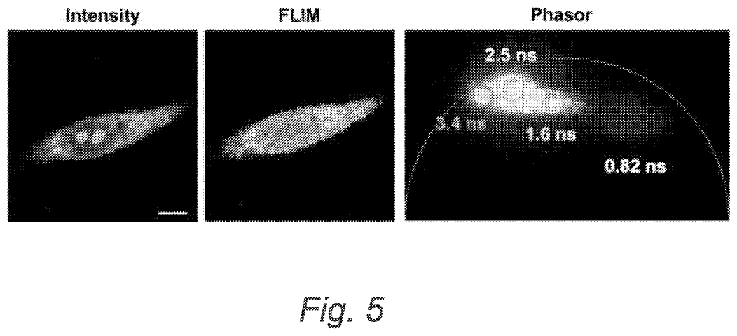

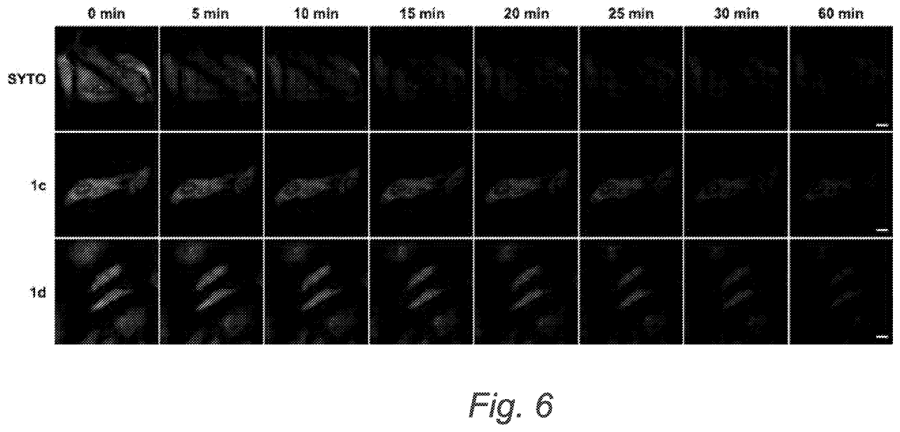

The cytotoxicity of 1c and 1d was evaluated by performing an MTT assay using HeLa cells. Cells were incubated with each dye at concentrations ranging from 0.1 to 30 M for 24 h. The results show that >70% of the cells remained viable after 24 h of incubation with 30 μM of either 1c or 1d. These indolizine-containing probes can thus be considered largely nontoxic for short-term imaging experiments. It is important to note that cytotoxicity results from repeated or prolonged exposure of fluorescently labelled cells to irradiation from high laser powers.38 Cells overexposed to irradiation may sustain damage to macromolecules and organelles which can negatively influence cell viability. Several studies have reported that red-shifted dyes are preferrable to shorter wavelength dyes (e.g., violet or blue excitation) due to the lower incidence of cell death.³⁹⁻⁴¹ To minimize toxicity, several factors must be considered: the dye concentration, laser power, and excitation wavelength. In the investigation, it was determined that 1c could be used at concentrations as low as 10 nM to resolve nucleoli while SYTO™ RNASelect required higher concentrations (>2 μM) to see fluorescent signals. Furthermore, the favorable photophysical properties of 1c (e.g., high quantum yield) allow for the facile acquisition of high-contrast live cell images with low laser power, while the same could not be achieved with SYTO. Finally, the excitation wavelength of both 1c and 1d are relatively more red-shifted compared to SYTO (Table 1). Having longer excitation wavelengths comes with many advantages including reduced photobleaching (FIG. 6), increased tissue penetration, and reduced autofluorescence.⁴²

The photostability of the new dyes was assessed compared to the commercially available RNA stain, SYTO. Fixed HeLa cells were incubated with 1 μM of 1c and SYTO for 30 min, thoroughly washed to remove excess dye. Cells were exposed to continuous irradiation under a fixed laser power and imaged over a range of different time points (FIG. 6). The fluorescence intensities at each time point was quantitatively analyzed and it was determined that 1c displayed significantly higher photostability, with a half-life (t1/2) of ~12.5 minutes; in contrast, SYTO displayed a t1/2 of ~1.5 minutes which is >8-fold less than that of 1c (FIG. 6).

### 2.5 Detection of RNA Coacervates Using 1c

Liquid-liquid phase separation (LLPS) has emerged as a new paradigm in the study of cellular processes.43 LLPS is thought to be the underlying mechanism behind the formation of intracellular membrane-less organelles (MLO) such as nucleoli and P granules.⁴³⁻⁴⁵ Studying these MLOs can provide insight into the molecular basis of disease.^{44,46} Thus, research efforts have focused on understanding their formation for further investigation of the physiology and pathophysiology of a wide range of biological processes and systems.⁴⁵ The most common method used to initially detect LLPS is microscopy. Hence, the use of fluorescently labeled condensate components can enable their detection in vitro and in cells. Previous studies have utilized fluorescently labelled RNA,^{47, 48} peptides,⁴⁹ and proteins⁵⁰⁻⁵² to visualize RNA coacervates, a type of droplet formed by LLPS. However, these strategies require modifying RNA and engineering proteins with exogenous fluorophores such as fluorescein and GFP. Given that 1c exhibits a remarkable fluorogenic response upon binding RNA, it is hypothesized that 1c could enrich and label RNA-coacervates. A simple in vitro model consisting of torula yeast RNA (negative polyelectrolyte) and spermine (positive polyelectrolyte) following an adapted procedure was devised.^{47, 48} The coacervates were incubated with 1c and imaged using confocal fluorescence microscopy and FLIM.

The formation of spherical coacervate droplets upon mixing the RNA and spermine solutions in a high ionic strength buffer was observed. Coacervates incubated with 1c were visualized using confocal fluorescence microscopy and FLIM (FIG. 7). Both intensity and FLIM images show that 1c readily partitions into RNA coacervates and exhibits intense fluorescence signals where RNA is densely concentrated. This is attributed to the cationic and lipophilic nature of 1c which favors accumulation in hydrophobic and water-poor regions while the strong fluorogenic response is due to reduced rotational freedom of the probe when bound to RNA. The phasor plot indicates the presence of a single fluorescence lifetime species which is evenly dispersed throughout the coacervate. In addition to the uniform coacervate droplets, multiphase complex coacervates are also formed under these conditions due to sufficient differences in macromolecular density driven by charge-charge interactions and critical salt concentrations.51 Interestingly, FLIM can better resolve the multiphase complex RNA coacervates (cavity-containing droplets) than that of the intensity-based imaging. The different layers in the coexisting phases present distinct chemical environments that can concentrate 1c or other guest molecules to different extents. The cavity observed in FIG. 7 is speculated to be a highly solvated region where fluorescence signals are quenched due to non-radiative decay. Further experiments would be needed to fully characterize the chemical environment of each phase.

### 3. Conclusion

In conclusion, a panel of fluorogenic styrene dyes for visualizing RNA in live cells and in RNA coacervates has been developed. Given that MPI has previously shown good cell permeability and RNA-selectivity, but displays only moderate fluorescence enhancement and quantum yield, analogs of MPI were generated to improve its spectral and photophysical properties while retaining its selectivity for RNA in cells. By changing the substitution position on the indole donor and replacing the indole with indolizine, four novel styrene dyes 1a-1d which exhibited significantly altered spectral and photophysical profiles were generated. Dyes 1a and 1b are mere regio-isomers of MPI yet they displayed >20 nm red shift in emission wavelengths and significantly larger Stokes shifts than MPI. The positional change of the indole donor from the 3- to 2-position present an altered electronic configuration that involves a greater area in the conjugated 7t-system of the dye scaffold and thus lowers the overall energy of the molecule. Apart from the significant, red-shifted emission wavelengths, dyes 1a and 1b displayed, on average, lower molar absorptivity and quantum yield values than that of MPI. Despite the excellent 104-fold fluorescence enhancement observed for 1a, its utility is limited due to its low quantum yield.

In contrast, when the indole donor was replaced with indolizine, the resulting indolizine-containing dyes exhibited improvements in not only the spectral properties but also the photophysical properties. These dyes were found to absorb and emit in the orange-red region of the visible spectrum, making them more ideal for imaging cells and tissues. Moreover, 1c and 1d displayed high quantum yields and a remarkable fluorescence enhancement upon binding RNA. It is reasoned that the donor replacement resulted in an expansion of conjugation in electron transfer to lower the overall energy of the system—similarly to what was observed for 1a and 1b. Rigorous computational studies are currently underway to explain the superior properties of indolizine over indole among these styrene dyes and will be published soon.

Indolizine is a nitrogen-containing heterocycle that has found many uses in medicinal chemistry and pharmaceuticals.^{53, 54} Recently, indolizines have been explored for applications in fluorescent and luminescent materials; notably in organic light-emitting diodes (OLEDs) because of its high quantum yield and tunable fluorescence properties.⁵⁵⁻⁵⁷ The experiments describe herein incorporates the indolizine moiety into a styrene scaffold for labeling RNA in live cells. The experiments validates the excellent photophysical properties of indolizines especially compared to its indole isostere. The experiments describe herein represents the first application of this interesting heterocycle in RNA-selective dyes for live cell labeling.

It has been demonstrated that 1c and 1d are compatible with both live- and fixed-cell imaging and can resolve sub-nucleolar structures such as the FC from the surrounding GC. Co-staining experiments with Hoechst 33342 suggests that the dyes are compatible with nuclear stains and are likely compatible with other organelle-specific dyes. The images show that 1c and 1d indeed label RNA-rich nucleoli with rapid labeling kinetics, high contrast and low background. Fluorescence signals are not observed in the surrounding nucleus suggesting that 1c and 1d do not bind to chromosomal DNA via intercalation or minor groove binding. Although the exact mechanism for the binding of these styrene dyes to RNA has not been explored; based on the observations it is believed that 1c and 1d may not function as classic intercalators or minor groove binders upon binding RNA. To further improve upon these dyes, efforts must be placed in determining the exact binding mode of these dyes to RNA.

In addition to cell-based imaging, it is show that the dyes can selectively partition and label RNA coacervates in vitro. Developing small molecule probes that can selectively accumulate in coacervates is instrumental in studying LLPS and can serve as key tools in delineating the function of biomolecular condensates in cells and their physiological and pathophysiological roles.

The dyes display good photostability and are noncytotoxic, making them useful for long-term, time resolved imaging experiments. The robust quantum yields of 1c and 1d negate the use of high laser powers to irradiate the dyes allowing for reduced incidence of phototoxicity. Given the high sensitivity of the dyes, concentrations as low as 10 nM was successfully used to resolve nucleoli. The new dyes display unique fluorescence lifetimes within distinct cellular and aqueous environments (i.e., nucleolus, nucleus, and cytoplasm) allowing for selective imaging based on fluorescence lifetimes. It is envisioned that the dyes to serve as excellent probes for FLIM experiments to resolve cellular structures beyond fluorescence intensity and emission wavelength. In fact, the dyes may even be compatible with other red-colored dyes (λem >580 nm) regardless of spectral overlap. Given the excellent fluorescence properties of the novel indolizine dyes, it is believed believe that they can serve as a better alternative to SYTO™ RNASelect in a wide variety of cell imaging studies.

### 4. Materials and Methods

4.1 Quantum Yield Measurements of 1a-1d in T.E. Buffer and RNA Solution

Coumarin 6 was used as a reference dye for MPI, 1a, and 1b and Rhodamine 6G was used as a reference for 1c and 1d. Absorption spectra were collected by Shimadzu UV-1800 spectrophotometer. Samples were loaded in plastic disposable cuvette. Emission spectra were obtained by Photon Technology International QuantaMaster model C-60 Fluorimeter in 1×1 cm² quartz cuvettes. Fluorescence quantum yields were then calculated according to the method by Lawson-wood, Upstone, and Evans. (58) The torula yeast type IV RNA (Sigma) solution was prepared as a 200 μg/mL solution in T.E. buffer (Tris-HCl, EDTA, pH 7.6, Bioworld) without sonicating to avoid shearing of nucleic acids. All measurements were taken using samples with a final dye concentration of 10 μM.

### 4.2 Fluorescence Titration of 1

Solutions of torula yeast RNA type IV (RNA mixture) and DNA from calf thymus (Sigma) were prepared and stored at 4° C. overnight. Bovine serum albumin (BSA, Thermo Fisher) and Baker's yeast RNA (rRNA, Sigma) solutions were prepared at 2 h before testing. 100 μL of 1 μM 1c were diluted by corresponding substrates solution reaching concentrations ranging from 0 to 1000 μg/mL of nucleic acids or BSA. The resulting mixtures were placed in a 96-well opaque plate. The plate with the mixture was gently shaken for 5 min before being evaluated by a microplate reader.

4.3 Comparison with SYTO RNASelect

100 μL of 1 μM 1c and SYTO RNASelect were diluted by 100 μL solutions of RNA mixture to reach final concentrations ranging from 0 to 1000 μg/mL. The resulting mixtures were placed in a 96-well opaque plate. The plate with the mixture was gently shaken for 5 min before being evaluated by a microplate reader (SpectraMax iD5).

### 4.4 General Method for Cell Culture

HeLa cells were cultured in Dulbecco's modified Eagle's medium (DMEM, Thermo Fisher) supplemented with 10% (v/v) fetal bovine serum (FBS, Thermo Fisher) and incubated at 37° C. with 5% CO₂.

### 4.5 Live-Cell Imaging

HeLa cells were cultured at a density of 7000 cells/well on chambered glass sides and incubated at 37° C. overnight or until fully adherent. After removing the medium and washing with DPBS (Thermo Fisher), the cells were incubated with dyes in PBS (Thermo Fisher) for 30 min at 37° C. with 5% CO₂ on the microscope stage and analyzed directly. Fluorescence images were acquired using a confocal microscope (Leica SP8, Leica Microsystems) and analyzed using ImageJ. MPI (20 μM) was excited at 440 nm, SYTO RNASelect (0.1-1 μM, Thermo Fisher) was excited at 490 nm, 1c (10 nM-20 μM) was excited at 550 nm, and 1d (20 μM) was excited at 500 nm.

### 4.6 Time-Resolved Live-Cell Imaging

HeLa cells were cultured on eight-well chambered glass slides (iBidi μ-slides) and incubated overnight at 37° C. with 5% CO₂ until totally adherent. Staining solutions for each dye were prepared in serum-free DMEM and kept warm at 37° C. The cells were monitored from 0 to 30 min, where t=0 is the time right before the addition of the dye. Images were acquired at t=0, 0.5, 1, 2, 5, 10, 15, 20, 25, and 30 min for each dye. Fluorescence images were analyzed and quantified with ImageJ.

### 4.7 Fixed-Cell Imaging

HeLa cells were cultured on 6-well plates containing glass coverslips and incubated at 37° C. overnight or until fully adherent. After removing the medium and washing with DPBS, the cells were fixed with 4% paraformaldehyde for 10 min at ambient temperature. After fixation, the cells were rinsed with PBS and incubated with dye solutions in PBS for 30 min at ambient temperature. After incubation, the dye solutions were removed and cells were gently washed with PBS three times. The coverslips were mounted onto glass slides, sealed, and analyzed using confocal microscopy.

### 4.8 Ribonuclease (RNase) Digest Experiment

HeLa cells were cultured in six-well plates with glass coverslips and incubated at 37° C. overnight to totally adherent. The cells were fixed according to the protocol described above with minor modifications. A solution of 4% paraformaldehyde+0.1% Triton-X was prepared in PBS and used to fix and permeabilize HeLa cells. The cells were incubated with this solution for 20 min at room temperature. After rinsing the cells with PBS, the cells were stained with 1 μM 1c, 1d, or SYTO in PBS for 30 min at room temperature. After removing the staining solution, 0.5 mL of RNase (100 g/mL) and PBS (negative control) was added into each respective well and incubated for 6 h. After incubation, the medium was removed, and the cells were thoroughly washed with PBS three times. The glass coverslips were mounted onto glass slides, sealed, and analyzed using confocal microscopy.

4.9 Counterstaining with Hoechst 33342

HeLa cells were cultured in six-well plates with glass coverslips and incubated at 37° C. overnight to totally adherent. The cells were fixed according to the protocol described above and stained with 20 M 1c or 1d and 1 μg/mL Hoechst 33342 (Thermo Fisher) diluted in PBS for 30 min in the dark. After incubation with dyes, the cells were gently washed with PBS. The coverslips were mounted onto glass slides, sealed, and analyzed using confocal microscopy.

### 4.10 Photostability

HeLa cells were fixed, stained with 1 M 1c and SYTO RNASelect, and mounted according to the protocol described above. The cells were analyzed using confocal microscopy and continuously irradiated at 550 nm (1c) and 490 nm (SYTO RNAselect) holding a fixed laser power. Images were taken at indicated time points (0, 5, 10, 15, 20, 25, 30, and 60 min). Fluorescence intensities were quantified using ImageJ.

### 4.11 In Vitro Assay for Cytotoxicity

HeLa cells were cultured (2000 cells/well) in DMEM and supplemented with 10% FBS in 96-well plates. The solution of MPI, 1c, and 1d at indicated concentrations (0, 0.1, 0.3 1, 3, 10, 30 μM) were added to each well diluted with DMEM and cells were incubated for 24 h at 37° C. with 5% CO₂. The next day, 10 μL of MTT (Abeam) labeling reagent was added to the pretreated cells and incubated for 4 h. A control was prepared in the same manner by adding 10 μL of MTT labeling reagent to untreated HeLa cells. After incubation, 100 μL of solubilization reagent was added to each well and the plate was shaken at 37° C. for 15 min in an orbital shaker. Upon complete solubilization of the purpose formazan crystals, the absorbance of the samples was measured at 570 nm using a microplate reader. The absorbance of each sample was normalized with its control. MTT assays were performed in triplicates.

### 4.12 RNA-Spermine Condensate Preparation

A 1 wt % stock solution of torula yeast RNA type IV was prepared in nuclease-free water and stored in multiple aliquots at −20° C. Final concentrations of RNA ranged from 0.02 to 0.4 wt %. A 1 wt % stock solution of spermine was prepared in deionized water and stored at 4° C. The spermine (Sigma) concentration was fixed at 0.1 wt %. Dye 1c was prepared as 10 mM stock solutions in DMSO. Condensates were prepared in a 5 mM HEPES (pH 7.4, VWR), 1 mM MgCl₂ buffer. Stock solutions were added in the following order for the preparation of each sample: deionized water, HEPES, MgCl₂, RNA, and spermine. Samples were mixed via gentle pipetting in between the addition of each component.

### 4.13 Staining and Imaging of Condensates

RNA condensates were incubated with 10 μM 1c for 1 min at room temperature. To image the condensates, 20 μL of the incubated sample was added onto a glass coverslip and mounted onto a glass slide. Images were acquired using fluorescence confocal microscopy and FLIM.

### 4.14 Native-PAGE Gel Staining

1 L of 10×TBE running buffer was prepared by mixing 108 g of Tris Base, 55 g of boric acid, and 40 mL of 0.5 M EDTA (Apex). The combined solution was diluted to 1 L by filtered H₂O. 14 mL of 8% Native-PAGE gel was prepared by mixing 0.7 mL of 10×TBE buffer, 0.14 mL of 10% APS buffer (Bio-Rad), and 3.73 mL of 30% acrylamide solution. The mixture was diluted to 14 mL with filtered water. 28 μL of TEMED (Sigma) was added to the mixture. The gel mixture was transferred into the casting frame placed in a precooled gel chamber. An 18-well cast was put on the top and left for polymerization for 15 min on ice. The gel chamber was filled with cold 0.5×TBE running buffer. The gel was pre-run at 150 V for 40 min. The RNA marker mixture was loaded (5 μL micro-RNA marker+0.4 μL low range marker (Bio-Rad) for one well). 5 μL of rRNA sample was loaded to reach rRNA amount as 1 and 10 μg. The RNA marker and rRNA sample loading had four repeats on the same gel. The gel was run at 150 V for 30 min. The cut gels were stained and shaken in 25 mL of 1×SYBR Gold (Thermo Fisher), 2 μM 1c, 20 μM 1c, and 40 μM 1c for 15 min. The gels were de-stained with 0.5×TBE buffer and shaken for 10 min. The buffer was removed, and the gels were imaged using an Amersham Typhoon Biomolecular Imnager.

### 5. Chemical Synthesis

### 5.1 Materials and Instruments

1H-indole-3-carbaldehyde and 1-methyl-1H-indole-3-carbaldehyde were purchased from 1PlusChem and ChemScene. 1-methyl-1H-indole-4-carbaldehyde, 1-methyl-1H-indole-5-carbaldehyde, 1-methyl-1H-indole-6-carbaldehyde, 1-methyl-1H-indole-7-carbaldehyde, 6-methylisoquinoline, 3-ethyl-2-methylbenzothiazolium iodide and 1-ethyl-2,3,3-trimethylindolenium iodide were purchased from Ambeed. 2-pyridiniumpropanol was purchased from AAblocks. 4-methylpyridine was purchased from TCI. Iodomethane and 10% palladium on Charcoal were purchased from Sigma. All organic solvents used in the synthesis were HPLC grade and purchased from Sigma Millipore.

Mass spectrometry or LC-MS was performed on a Finnigan LCQ Deca XP Max equipped with an electrospray ionization (ESI) source (positive ion mode) and a photodiode array (PDA) detector.

### 5.2 Synthetic Procedures

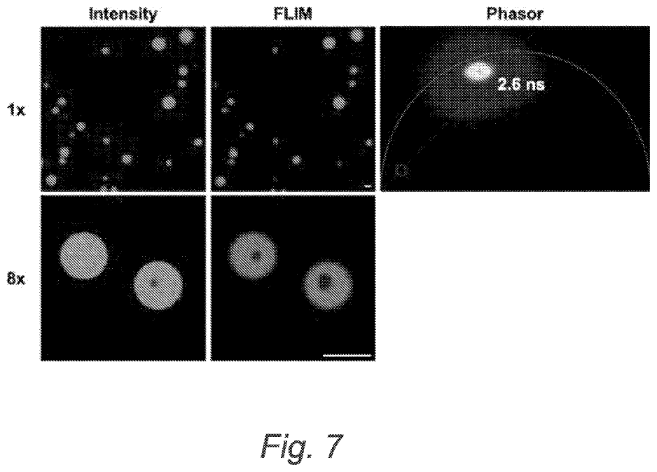

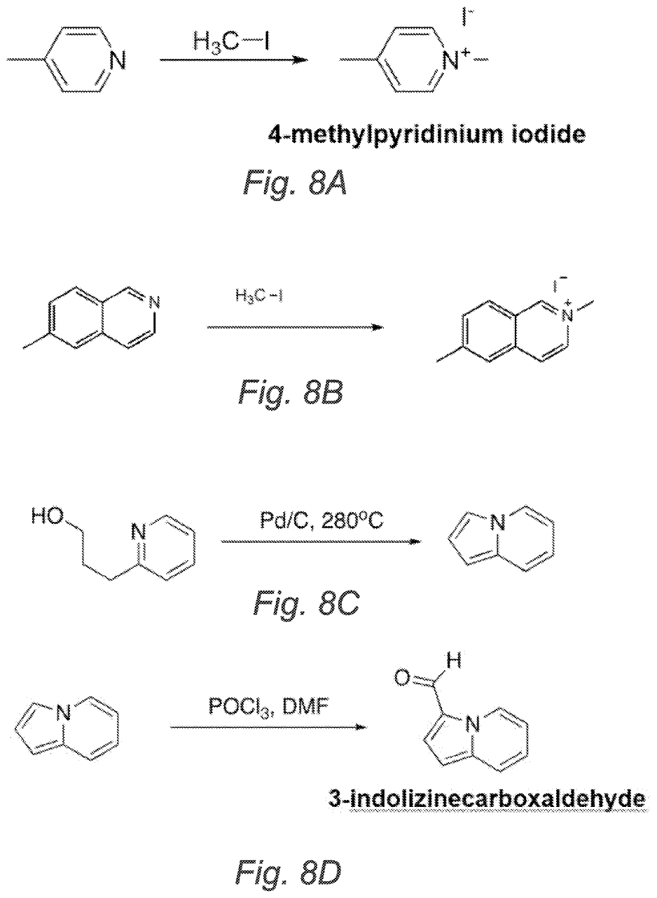

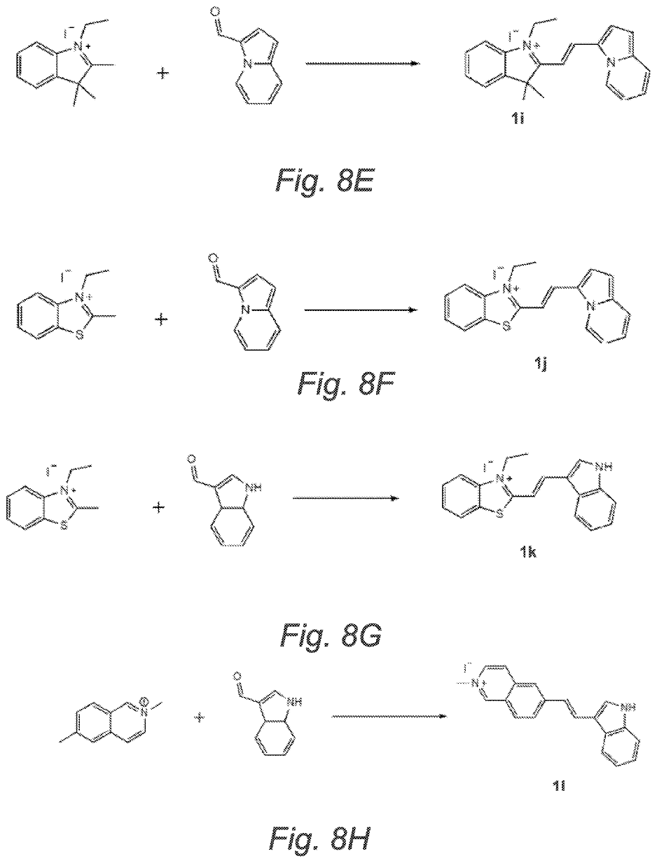

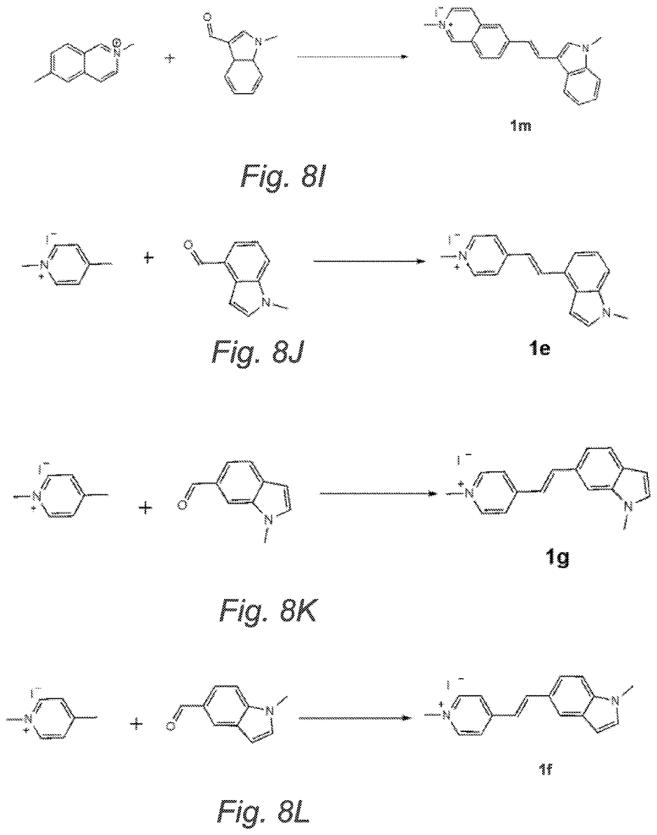

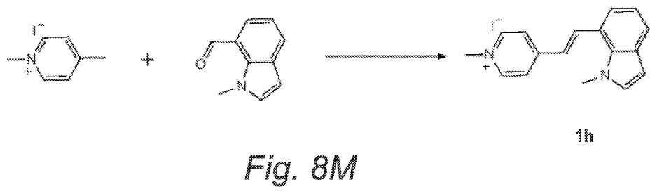

1,4-dimethylpyridinium iodide. 4-Methylpyridine (10.2 mmol) was added to iodomethane (32 mmol) solution in Et₂O with stirring for 2 hours at room temperature. The product as solid was collected by vacuum filtration and used in subsequent reactions without further characterization. (FIG. 8A).

2,6-dimethylquinolinium iodide. 6-Methylquinoline (1 mmol) was added to iodomethane (3 mmol) solution in acetonitrile (2 mL) with stirring at 80° C. for 16 hours to afford 2,6-dimethylquinolinium iodide with 80% yield. The product as solid was collected by vacuum filtration, washed with ethyl acetate used in subsequent reactions without further characterization. (FIG. 8B).

Indolizine. A mixture of 2-pyridinepropanol (1 mL, 7.27 mmol) and palladium on activated carbon (10%) (15 mg, 10 mmol %) was refluxed under N₂ atmosphere at 280° C. for 8 hours. The residue was filtered and washed with MeOH. The filtrate was collected and concentrated in vacuo. The solution was purified by Flash chromatography to afford 111 mg of a white solid in 13% yield. The product was used in subsequent reactions without further characterization³. (FIG. 8C).

3-indolizinecarboxaldehyde. To a stirred solution of indolizine (0.1 g, 0.93 mmol) in 1.2 mL DMF was added dropwise a 3 M POCl₃ solution in DMF. The mixture was stirred for 2 hours at room temperature. After stirring, 1.5 mL of ice water and 1.1 mL of 25% NaOH was added. The mixture was refluxed for 10 minutes. The filtrate was extracted with chloroform and dried over Na₂CO₃ and concentrated in vacuo. The concentrated mixture was purified by flash chromatography to afford 63 mg of compound 2c as dark-blue oil in 47% yield⁴. (FIG. 8D).

General procedure A: A solution mixture of iodide salt (0.22 mmol), aldehyde (0.2 mmol) and piperidine (0.02 mL) in EtOH (2 mL) was refluxed at 70° C. overnight. After cooling down to room temperature, the residue was filtered, washed with cold ethanol to afford the product.

Dye 1i. 1-Ethyl-2,3,3-trimethylindolenium iodide and 3-indolizinecarboxaldehyde were used according to general procedure A to afford the product with 40% yield. LC-MS (ESI) (m/z) calculated for C₂₂H₂₃N₂⁺ [M⁺H]⁺ 315.19, found 315.19. (FIG. 8E).

Dye 1j. 3-Ethyl-2-methylbenzothiazolium iodide and 3-indolizinecarboxaldehyde were used according to general procedure A to afford the product with 32% yield. LC-MS (ESI) (m/z) calculated for C₁₉H₁₇N₂S⁺ [M⁺H]⁺ 305.11, found 305.11. (FIG. 8F).

General procedure B: A solution mixture of iodide salt (0.5 mmol), aldehyde (0.08 g, 0.5 mmol) and piperidine (0.07 mL) in MeOH (5 mL) was refluxed at 60° C. overnight. After cooling down to room temperature, the residue was filtered, washed with cold methanol to afford the product. (FIG. 8G).

Dye 1k. 3-Ethyl-2-methylbenzothiazolium iodide and 1H-indole-3-carbaldehyde were used according to general procedure B to afford the product with 13% yield. LC-MS (ESI) (m/z) calculated for C₁₉H₁₇N₂S⁺ [M⁺H]⁺ 305.11, found 305.11. (FIG. 8G).

Dye 11. 2,6-Dimethylquinolinium iodide and 1H-indole-3-carbaldehyde were used according to general procedure B to afford the product with 2% yield. LC-MS (ESI) (n/z) calculated for C₂₀H₁₇N₂⁺ [M⁺H]⁺ 285.37, found 285.14. (FIG. 8H).

Dye 1m. 2,6-Dimethylquinolinium iodide and 1-methyl-1H-indole-3-carbaldehyde were used according to general procedure B to afford the product with 24% yield. LC-MS (ESI) (m/z) calculated for C₂₁H₁₉N₂⁺ [M⁺H]⁺ 299.15, found 299.16. (FIG. 8I).

Dye 1e 1,4-Dimethylpyridinium iodide and 1-methyl-1H-indole-4-carbaldehyde were used according to general procedure B to afford the product with 64% yield. LC-MS (ESI) (m/z) calculated for C₁₇H₁₇N₂⁺ [M⁺H]⁺ 249.34, found 249.14. (FIG. 8J).

Dye 1g. 1,4-Dimethylpyridinium iodide and 1-methyl-1H-indole-5-carbaldehyde were used according to general procedure B to afford the product with 14% yield. LC-MS (ESI) (m/z) calculated for C₁₇H₁₇N₂⁺ [M⁺H]⁺ 249.34, found 249.14. (FIG. 8K).

Dye 1f. 1,4-Dimethylpyridinium iodide and 1-methyl-1H-indole-6-carbaldehyde were used according to general procedure B to afford the product with 66% yield. LC-MS (ESI) (m/z) calculated for C₁₇H₁₇N₂⁺ [M⁺H]⁺ 249.34, found 249.14. (FIG. 8L).

Dye 1h. 1,4-Dimethylpyridinium iodide and 1-methyl-1H-indole-7-carbaldehyde were used according to general procedure B to afford the product with 8% yield. LC-MS (ESI) (m/z) calculated for C₁₇H₁₇N₂⁺ [M⁺H]⁺ 249.34, found 249.14. (FIG. 8M).

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. Schulz, D.; Holstein, J. M.; Rentmeister, A., A chemo-enzymatic approach for site-specific modification of the RNA cap. Angew Chem Int Ed Engl 2013, 52 (30), 7874-8.
2. Braselmann, E.; Rathbun, C.; Richards, E. M.; Palmer, A. E., Illuminating RNA Biology: Tools for Imaging RNA in Live Mammalian Cells. Cell Chem Biol 2020, 27 (8), 891-903.
3. Antony, T.; Subramaniam, V., Molecular beacons: nucleic acid hybridization and emerging applications. J Biomol Struct Dyn 2001, 19 (3), 497-504.
4. Soboleski, M. R.; Oaks, J.; Halford, W. P., Green fluorescent protein is a quantitative reporter of gene expression in individual eukaryotic cells. FASEB J 2005, 19 (3), 440-2.
5. Cui, C.; Shu, W.; Li, P., Fluorescence In situ Hybridization: Cell-Based Genetic Diagnostic and Research Applications. Front Cell Dev Biol 2016, 4, 89.
6. Strack, R. L.; Song, W.; Jaffrey, S. R., Using Spinach-based sensors for fluorescence imaging of intracellular metabolites and proteins in living bacteria. Nat Protoc 2014, 9 (1), 146-55.
7. Muttach, F.; Muthmann, N.; Rentmeister, A., Chemo-enzymatic modification of eukaryotic mRNA. Org Biomol Chem 2017, 15 (2), 278-284.
8. Paige, J. S.; Wu, K. Y.; Jaffrey, S. R., RNA mimics of green fluorescent protein. Science 2011, 333 (6042), 642-6.
9. Bao, G.; Rhee, W. J.; Tsourkas, A., Fluorescent probes for live-cell RNA detection. Annu Rev Biomed Eng 2009, 11, 25-47.
10. Kubota, T.; Ikeda, S.; Yanagisawa, H.; Yuki, M.; Okamoto, A., Sets of RNA repeated tags and hybridization-sensitive fluorescent probes for distinct images of RNA in a living cell. PLoS One 2010, 5 (9), e13003.
11. Catrina, I. E.; Marras, S. A.; Bratu, D. P., Tiny molecular beacons: LNA/2′-O-methyl RNA chimeric probes for imaging dynamic mRNA processes in living cells. ACS Chem Biol 2012, 7 (9), 1586-95.
12. Holstein, J. M.; Stummer, D.; Rentmeister, A., Enzymatic modification of 5′-capped RNA with a 4-vinylbenzyl group provides a platform for photoclick and inverse electron-demand Diels-Alder reaction. Chem Sci 2015, 6 (2), 1362-1369.
13. Wang, L.; Frei, M. S.; Salim, A.; Johnsson, K., Small-Molecule Fluorescent Probes for Live-Cell Super-Resolution Microscopy. J Am Chem Soc 2019, 141 (7), 2770-2781.
14. Lavis, L. D.; Raines, R. T., Bright ideas for chemical biology. ACS Chem Biol 2008, 3 (3), 142-55.
15. Yao, Q.; Li, H.; Xian, L.; Xu, F.; Xia, J.; Fan, J.; Du, J.; Wang, J.; Peng, X., Differentiating RNA from DNA by a molecular fluorescent probe based on the “door-bolt” mechanism biomaterials. Biomaterials 2018, 177, 78-87.
16. Li, Q.; Kim, Y.; Namm, J.; Kulkarni, A.; Rosania, G. R.; Ahn, Y. H.; Chang, Y. T., RNA-selective, live cell imaging probes for studying nuclear structure and function. Chem Biol 2006, 13 (6), 615-23.
17. Wang, H.; Tian, X.; Du, W.; Zhang, Q.; Guan, L.; Wang, A.; Zhang, Y.; Wang, C.; Zhou, H.; Wu, J.; Tian, Y., A twophoton fluorescent RNA probe screened from a series of oxime-functionalized 2,2′:6′,2″-terpyridine ZnX2 (X=Cl, Br, I) complexes. J Mater Chem B 2016, 4 (28), 4818-4825.
18. Liu, Y.; Meng, F.; He, L.; Yu, X.; Lin, W., Fluorescence behavior of a unique two-photon fluorescent probe in aggregate and solution states and highly sensitive detection of RNA in water solution and living systems. Chem Commun (Camb) 2016, 52 (57), 8838-41.
19. Guo, L.; Chan, M. S.; Xu, D.; Tam, D. Y.; Bolze, F.; Lo, P. K.; Wong, M. S., Indole-based cyanine as a nuclear RNAselective two-photon fluorescent probe for live cell imaging. ACS Chem Biol 2015, 10 (5), 1171-5.
20. Song, G.; Sun, Y.; Liu, Y.; Wang, X.; Chen, M.; Miao, F.; Zhang, W.; Yu, X.; Jin, J., Low molecular weight fluorescent probes with good photostability for imaging RNA-rich nucleolus and RNA in cytoplasm in living cells. Biomaterials 2014, 35 (7), 2103-12.
21. Grimm, J. B.; Heckman, L. M.; Lavis, L. D., The chemistry of small-molecule fluorogenic probes. Prog Mol Biol Transl Sci 2013, 113, 1-34.
22. Boekelheide, V.; Windgassen, R. J., Syntheses of Pyrrocolines Unsubstituted in the Five-membered Ring. J Am Chem Soc 1959, 81 (6), 1456-1459.
23. Fuentes, O.; Paudler, W. W., Some formylation reactions of imidazo[1,5-a]pyridine and pyrrocoline. Journal of Heterocyclic Chemistry 1975, 12, 379-393.
24. Olsen, S., Locally-excited (LE) versus charge-transfer (CT) excited state competition in a series of para-substituted neutral green fluorescent protein (GFP) chromophore models. J Phys Chem B 2015, 119 (6), 2566-75.
25. Shank, N. I.; Pham, H. H.; Waggoner, A. S.; Armitage, B. A., Twisted cyanines: a non-planar fluorogenic dye with superior photostability and its use in a protein-based fluoromodule. J Am Chem Soc 2013, 135 (1), 242-51.
26. Silva, G. L.; Ediz, V.; Yaron, D.; Armitage, B. A., Experimental and computational investigation of unsymmetrical cyanine dyes: understanding torsionally responsive fluorogenic dyes. J Am Chem Soc 2007, 129 (17), 5710-8.
27. Ediz, V.; Lee, J. L.; Armitage, B. A.; Yaron, D., Molecular engineering of torsional potentials in fluorogenic dyes via electronic substituent effects. J Phys Chem A 2008, 112 (40), 9692-701.
28. Schleper, A. L.; Goushi, K.; Bannwarth, C.; Haehnle, B.; Welscher, P. J.; Adachi, C.; Kuehne, A. J. C., Hot exciplexes in U-shaped TADF molecules with emission from locally excited states. Nat Commun 2021, 12 (1), 6179.
29. Ha, J. M.; Hur, S. H.; Pathak, A., Recent advances in organic luminescent materials with narrowband emission. NPG Asia Mater 2021, 13 (53).
30. Sivandzade, F.; Bhalerao, A.; Cucullo, L., Analysis of the Mitochondrial Membrane Potential Using the Cationic JC-1 Dye as a Sensitive Fluorescent Probe. Bio Protoc 2019, 9 (1).
31. Nodling, A. R.; Mills, E. M.; Li, X.; Cardella, D.; Sayers, E. J.; Wu, S. H.; Jones, A. T.; Luk, L. Y. P.; Tsai, Y. H., Cyanine dye mediated mitochondrial targeting enhances the anti-cancer activity of small-molecule cargoes. Chem Commun (Camb) 2020, 56 (34), 4672-4675.
32. Dove, B. K.; You, J. H.; Reed, M. L.; Emmett, S. R.; Brooks, G.; Hiscox, J. A., Changes in nucleolar morphology and proteins during infection with the coronavirus infectious bronchitis virus. Cell Microbiol 2006, 8 (7), 1147-57.
33. Cerqueira, A. V.; Lemos, B., Ribosomal DNA and the Nucleolus as Keystones of Nuclear Architecture, Organization, and Function. Trends Genet 2019, 35 (10), 710-723.
34. Ide, S.; Imai, R.; Ochi, H.; Maeshima, K., Transcriptional suppression of ribosomal DNA with phase separation. Sci Adv 2020, 6 (42).
35. Thiry, M.; Lafontaine, D. L., Birth of a nucleolus: the evolution of nucleolar compartments. Trends Cell Biol 2005, 15 (4), 194-9.
36. Digman, M. A.; Caiolfa, V. R.; Zamai, M.; Gratton, E., The phasor approach to fluorescence lifetime imaging analysis. Biophys J 2008, 94 (2), L14-6.
37. Battisti, A.; Digman, M. A.; Gratton, E.; Storti, B.; Beltram, F.; Bizzarri, R., Intracellular pH measurements made simple by fluorescent protein probes and the phasor approach to fluorescence lifetime imaging. Chem Commun (Camb) 2012, 48 (42), 5127-9.
38. Icha, J.; Weber, M.; Waters, J. C.; Norden, C., Phototoxicity in live fluorescence microscopy, and how to avoid it. Bioessays 2017, 39 (8).
39. Waldchen, S.; Lehmann, J.; Klein, T.; van de Linde, S.; Sauer, M., Light-induced cell damage in live-cell superresolution microscopy. Sci Rep 2015, 5, 15348.
40. Douthwright, S.; Sluder, G., Live Cell Imaging: Assessing the Phototoxicity of 488 and 546 nm Light and Methods to Alleviate it. J Cell Physiol 2017, 232 (9), 2461-2468.
41. Kilian, N.; Goryaynov, A.; Lessard, M. D.; Hooker, G.; Toomre, D.; Rothman, J. E.; Bewersdorf, J., Assessing photodamage in live-cell STED microscopy. Nat Methods 2018, 15 (10), 755-756.
42. Chica, R. A.; Moore, M. M.; Allen, B. D.; Mayo, S. L., Generation of longer emission wavelength red fluorescent proteins using computationally designed libraries. Proc Natl Acad Sci USA 2010, 107 (47), 20257-62.
43. Babinchak, W. M.; Dumm, B. K.; Venus, S.; Boyko, S.; Putnam, A. A.; Jankowsky, E.; Surewicz, W. K., Small molecules as potent biphasic modulators of protein liquid-liquid phase separation. Nature Communications 2020, 11 (1), 5574.
44. Alberti, S.; Gladfelter, A.; Mittag, T., Considerations and Challenges in Studying Liquid-Liquid Phase Separation and Biomolecular Condensates. Cell 2019, 176 (3), 419-434.
45. Lu, J.; Qian, J.; Xu, Z.; Yin, S.; Zhou, L.; Zheng, S.; Zhang, W., Emerging Roles of Liquid-Liquid Phase Separation in Cancer: From Protein Aggregation to Immune-Associated Signaling. Front Cell Dev Biol 2021, 9, 631486.
46. Alberti, S.; Hyman, A. A., Biomolecular condensates at the nexus of cellular stress, protein aggregation disease and ageing. Nat Rev Mol Cell Biol 2021, 22 (3), 196-213.
47. Aumiller, W. M., Jr.; Pir Cakmak, F.; Davis, B. W.; Keating, C. D., RNA-Based Coacervates as a Model for Membraneless Organelles: Formation, Properties, and Interfacial Liposome Assembly. Langmuir 2016, 32 (39), 10042-10053.
48. Marianelli, A. M.; Miller, B. M.; Keating, C. D., Impact of macromolecular crowding on RNA/spermine complex coacervation and oligonucleotide compartmentalization. Soft Matter 2018, 14 (3), 368-378.
49. Niu, J.; Qiu, C.; Abbott, N. L.; Gellman, S. H., Formation of versus Recruitment to RNA-Rich Condensates: Controlling Effects Exerted by Peptide Side Chain Identity. J Am Chem Soc 2022, 144(23), 10386-10395.
50. Lu, T.; Liese, S.; Schoenmakers, L.; Weber, C. A.; Suzuki, H.; Huck, W. T. S.; Spruijt, E., Endocytosis of Coacervates into Liposomes. J Am Chem Soc 2022, 144 (30), 13451-13455.
51. Lu, T.; Spruijt, E., Multiphase Complex Coacervate Droplets. J Am Chem Soc 2020, 142 (6), 2905-2914.
52. Patel, A.; Lee, H. O.; Jawerth, L.; Maharana, S.; Jahnel, M.; Hein, M. Y.; Stoynov, S.; Mahamid, J.; Saha, S.; Franzmann, T. M.; Pozniakovski, A.; Poser, I.; Maghelli, N.; Royer, L. A.; Weigert, M.; Myers, E. W.; Grill, S.; Drechsel,
D.; Hyman, A. A.; Alberti, S., A Liquid-to-Solid Phase Transition of the ALS Protein FUS Accelerated by Disease Mutation. Cell 2015, 162 (5), 1066-77.
53. Shen, Y. M.; Lv, P. C.; Chen, W.; Liu, P. G.; Zhang, M. Z.; Zhu, H. L., Synthesis and antiproliferative activity of indolizine derivatives incorporating a cyclopropylcarbonyl group against Hep-G2 cancer cell line. Eur J Med Chem 2010, 45 (7), 3184-90.
54. Sharma, V.; Kumar, V., Indolizine: a biologically active moiety. Medicinal Chemistry Research 2014, 23 (8), 3593-3606.
55. Song, Y. R.; Lim, C. W.; Kim, T. W., Synthesis and photophysical properties of 1,2-diphenylindolizine derivatives: fluorescent blue-emitting materials for organic light-emitting device. Luminescence 2016, 31 (2), 364-371.
56. Wan, J.; Zheng, C.-J.; Fung, M.-K.; Liu, X.-K.; Lee, C.-S.; Zhang, X.-H., Multifunctional electron-transporting indolizine derivatives for highly efficient blue fluorescence, orange phosphorescence host and two-color based white OLEDs. Journal of Materials Chemistry 2012, 22 (10), 4502-4510.
57. Kim, T.; Kim, J. A.-O., Color-Tunable Indolizine-Based Fluorophores and Fluorescent pH Sensor. LID-10.3390/molecules27010012 [doi] LID-12. (1420-3049 (Electronic)).

1. A fluorogenic styrene analog probe composition for visualizing RNA comprising a styrene analog having formula 1: 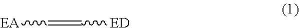
wherein:
the styrene analog having formula 1 is cis or trans;
EA is an electron acceptor selected from the group consisting of: 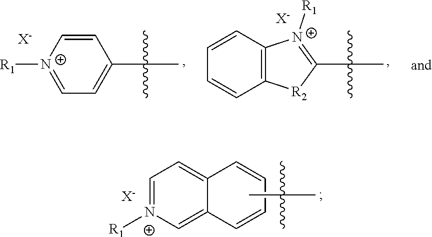
ED is an electron donor selected from the group consisting of: 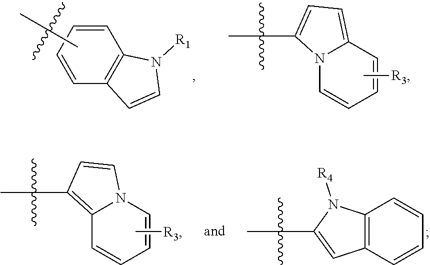
R₁ is H or C₁₋₈ alkyl;
R₂ is S or C(R₅)₂;
R₃ is H, C₁₋₈ alkyl, halo, C₁₋₈ alkoxy, —NH₂, —N(R₃)₂, or piperidinyl; and
R₄ is H or C₁₋₈ alkyl; and
R₅ is C₁₋₈ alkyl.

2. The fluorogenic styrene analog probe composition of claim 1, wherein the styrene analog has formula 1a:

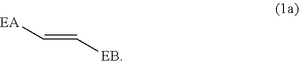

3. The fluorogenic styrene analog probe composition of claim 1, wherein EA is an electron acceptor having the following formula:

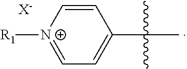

4. The fluorogenic styrene analog probe composition of claim 3 comprising a component selected from the group consisting of:

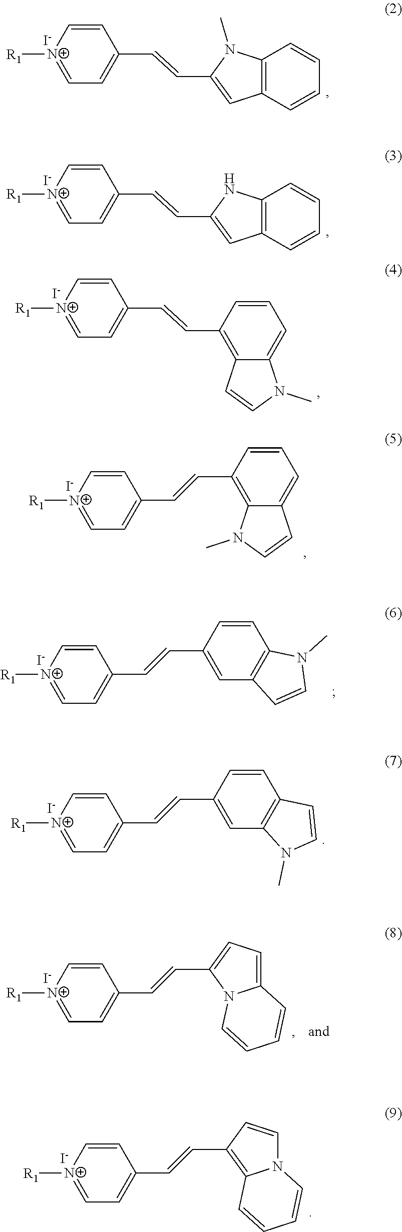

5. The fluorogenic styrene analog probe composition of claim 1, wherein EA is an electron acceptor having the following formula:

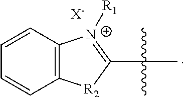

6. The fluorogenic styrene analog probe composition of claim 5 comprising a component selected from the group consisting of:

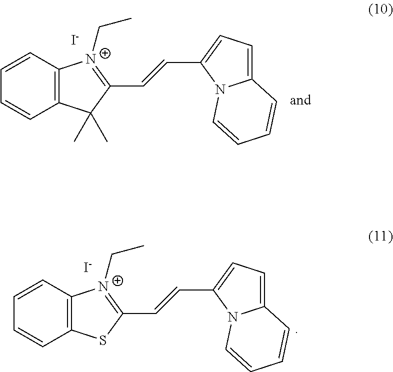 [SMILES: CC[N+]1=C(/C=C/c2ccc3ccccn23)C(C)(C)c2ccccc21 ; [I-] ; CC[n+]1c(/C=C/c2ccc3ccccn23)sc2ccccc21 ; [I-]]

7. The fluorogenic styrene analog probe composition of claim 1 further comprising a solvent.

8. The fluorogenic styrene analog probe composition of claim 7, wherein the styrene analog has a solubility greater than 100 uM in the solvent.

9. The fluorogenic styrene analog probe composition of claim 7, wherein the styrene analog has a solubility from the styrene analog has a solubility from 1 μM to 50 μM in the solvent.

10. The fluorogenic styrene analog probe composition of claim 1 providing red-shifted absorption and emission relative to methyl pyridinium indole.

11. A fluorogenic styrene analog probe composition for visualizing RNA comprising a styrene analog having formula 1: 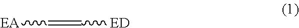
wherein:
the styrene analog having formula 1 is cis or trans;
EA is an electron acceptor selected from the group consisting of: 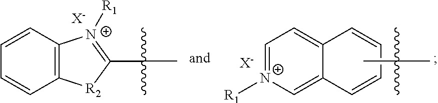
ED is an electron donor having the following formula: 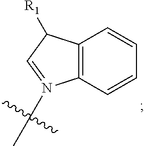
R₁ is H or C₁₋₈ alkyl, R₂ is S or C(R₅)₂;
R₃ is H, C₁₋₈ alkyl, halo, C₁₋₈ alkoxy, —NH₂, —N(R₃)₂, or piperidinyl; and
R₅ is C₁₋₈ alkyl.

12. The fluorogenic styrene analog probe composition of claim 11, wherein EA is an electron acceptor having the following formula:

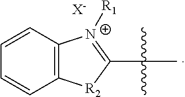

13. The fluorogenic styrene analog probe composition of claim 12 comprising a component selected from the group consisting of:

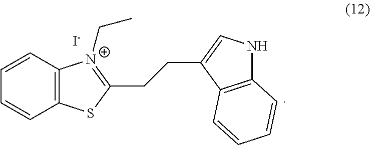 [SMILES: CC[n+]1c(CCc2c[nH]c3ccccc23)sc2ccccc21 ; [I-]]

14. The fluorogenic styrene analog probe composition of claim 11, wherein EA is an electron acceptor having the following formula:

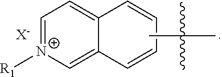

15. The fluorogenic styrene analog probe composition of claim 14 comprising a component selected from the group consisting of:

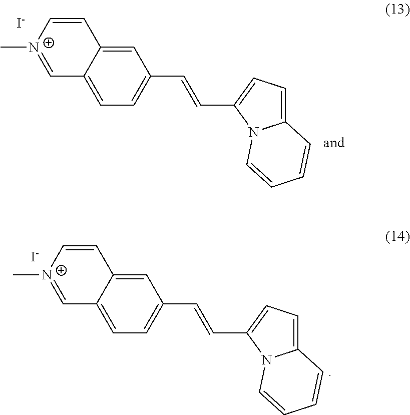 [SMILES: C[n+]1ccc2cc(/C=C/c3ccc4ccccn34)ccc2c1 ; [I-] ; C[n+]1ccc2cc(/C=C/c3ccc4ccccn34)ccc2c1 ; [I-]]

16. A method for imaging RNA is a biological sample, the method comprising:
contacting the biological sample with a styrene analog with a fluorogenic styrene analog probe composition for visualizing RNA to form a stained sample, the fluorogenic styrene analog probe composition comprising a styrene analog of claim 1; and
applying a fluorescence imaging technique to visualize the stained sample.

17. The method of claim 16, where the fluorescence imaging technique is selected from the group consisting of confocal microscopy, two-photon microscopy, fluorescence lifetime imaging microscopy (FLIM), and super-resolution microscopy.

18. The method of claim 16, wherein the fluorescence imaging technique is confocal fluorescence microscopy.

19. The method of claim 16, wherein the fluorescence imaging technique is fluorescence lifetime imaging microscopy.

20. The method of claim 16 wherein the method is compatible with live- and fixed-cell imaging.

21. The method of claim 16, wherein the fluorogenic styrene analog probe is incubated with the biological sample for a period ranging from 5 to 60 minutes before imaging.

22. The method of claim 16, further comprising a washing step after the contacting step to remove unbound fluorogenic styrene analog probe from the biological sample before applying the fluorescence imaging technique.

23. The method of claim 16, wherein the biological sample is selected from a group consisting of cultured cell lines, tissue sections, and single-cell suspensions.

24. The method of claim 16, wherein the fluorogenic styrene analog probe composition is applied to the biological sample at a concentration ranging from 1 μM to 50 μM.

25. The method of claim 16, wherein the fluorescence imaging technique includes the use of a specific wavelength for excitation and emission that maximizes a fluorescence signal from the fluorogenic styrene analog probe.

26. The method of claim 16, further comprising quantifying the RNA based on a fluorescence intensity measured from the stained sample.

27. A live-cell RNA imaging kit comprising:
one or more fluorogenic styrene probes of any of claim 1; and
a set of control probes that do not bind RNA, for use as negative controls in fluorescence imaging.

28. The live-cell RNA imaging kit of claim 27 further comprising instructions for staining live cells with the fluorogenic styrene probes and guidelines for fluorescence imaging, including suggested incubation times at least 5 minutes and concentrations for optimal RNA visualization.

29. The live-cell RNA imaging kit of claim 27, wherein the set of control probes includes phosphate-buffered saline.

30. The live-cell RNA imaging kit of claim 27, wherein the suggested incubation times is at least 5 minutes.
